(19) United States
(12) Patent Application Publication
(10) Pub. No.: US 2025/0368711 A1
(43) Pub. Date: Dec. 4, 2025

(54) COMPOSITIONS AND METHODS FOR TREATING MEIBOMIAN GLAND DYSFUNCTION

(71) Applicant: Cavalry Biosciences, Inc., San Francisco, CA (US)

(72) Inventors: Nathaniel Eames DAVID, San Francisco, CA (US); Robert Norman O'BRIEN, Burlington, VT (US); Timothy R. STOWE, San Francisco, CA (US); Jan VAN DEURSEN, Novato, CA (US); James W. WEST, Bend, OR (US); Patrick Brit VENTURA, San Francisco, CA (US); Janine Hendrike VAN REE, Novato, CA (US)

(21) Appl. No.: 19/299,180

(22) Filed: Aug. 13, 2025

Related U.S. Application Data
(63) Continuation of application No. 19/242,613, filed on Jun. 18, 2025.
Application No. 19/242,613 is a continuation of application No. PCT/US2023/085559, filed on Dec. 21, 2023.
(60) Provisional application No. 63/476,808, filed on Dec. 22, 2022.

(51) Int. Cl. C07K 14/65; A61K 9/00; A61K 38/00; A61P 27/02

(52) U.S. Cl. CPC C07K 14/65; A61K 9/0048; A61P 27/02; A61K 38/00

(57) ABSTRACT

The present disclosure provides composition for use in treating eye-related disorders, such as disorders affecting Meibomian glands. Composition for use in such disorders may act on the IGF-1 pathway, such as by binding to (e.g., agonizing) IGF1R. Other compositions may act on the IGF-1 pathway via another mechanism. Related methods, kits, and pharmaceutical compositions for treating eye-related disorders are also disclosed.

### CROSS-REFERENCE

This application is a continuation of U.S. patent application Ser. No. 19/242,613, filed Jun. 18, 2025, which is a continuation of International Patent Application No. PCT/US2023/085559, filed Dec. 21, 2023, which claims the benefit of U.S. Provisional Application No. 63/476,808, filed on Dec. 22, 2022, which application is incorporated herein by reference in its entirety.

### SEQUENCE LISTING

The instant application contains a Sequence Listing which has been submitted electronically in XML file format and is hereby incorporated by reference in its entirety. Said XML copy, created on August 13, is named 61766-715_302_SL.xml and is 12,698 bytes in size.

### BACKGROUND OF THE INVENTION

Meibomian glands are oil glands located primarily along the edge of the upper and lower eyelids. These glands serve a significant role in preventing dryness in eyes: they secrete meibum, a lipid and protein mixture that coats the surface of eyes, and prevent evaporation of the aqueous component of tears. Dysfunction and changes in the morphology of meibomian glands can lead to dry eye disease (DED). There are no approved medicines to treat Meibomian Gland Dysfunction (MGD). While there are medications that address a handful of the symptoms, no disease-modifying therapy exists today. The approved drugs target inflammation or drive the production of low-quality aqueous tears but do not deliver any long-term improvement to the patient. In addition, these therapies all require frequent dosing and high patient compliance to have any effect at all.

### SUMMARY OF THE INVENTION

In an aspect, the present disclosure provides a pharmaceutical composition comprising a therapeutically effective amount of an IGF-1 variant that has reduced affinity to at least one IGF binding protein (IGFBP) as compared to the affinity for the interaction between wild-type IGF-1 (SEQ ID NO: 1) and the IGFBP, wherein the pharmaceutical composition is formulated for local administration. In some embodiments, the pharmaceutical composition is formulated for local administration to an eye or eyelid.

In another aspect, the present disclosure provides a pharmaceutical composition comprising a therapeutically effective amount of an IGF-1 variant, wherein a local administration of the pharmaceutical composition to an eye or eyelid results in one or more of an increase in a size of meibomian glands; a decrease in meibomian gland atrophy; a reversal of age-associated meibomian gland atrophy; an increase in a function of one or more meibocytes; an increase in corneal epithelial cell proliferation; an increase in corneal healing rate; an increase in IGF1 receptor (IGF1R) activation in the meibomian glands; an increase duration of IGF1R activation in the meibomian glands; and an increase of lipid content of the meibomian glands.

In some embodiments, the IGF-1 variant has reduced affinity to at least one IGF binding protein relative to wild-type IGF-1 to the IGFBP. In some embodiments, the IGF-1 variant has at least 80%, at least 85%, at least 90%, at least 95%, or at least 98% sequence identity to SEQ ID NO: 1. In some embodiments, the pharmaceutical composition is formulated for delivery via an eyedrop. In some embodiments, the pharmaceutical composition comprises a cream for administration to one or both eyelids of a subject. In some embodiments, the pharmaceutical composition, when administered to subjects suffering from Meibomian gland dysfunction, results in a median increase in surface area or volume of meibomian glands within the inner eyelid surface of the subject as compared to subjects who do not receive the pharmaceutical composition. In some embodiments, the pharmaceutical composition, when administered to subjects suffering from Meibomian gland dysfunction, results in a median increase in lipid content within a Meibomian gland of the subject as compared to subjects who do not receive the pharmaceutical composition. In some embodiments, the pharmaceutical composition, when administered to subjects suffering from Meibomian gland dysfunction results, in a median increase in lipid quality within a Meibomian gland of the subject as compared to subjects who do not receive the pharmaceutical composition. In some embodiments, the pharmaceutical composition, when administered to subjects suffering from Meibomian gland dysfunction, results in a median increase in release of lipid from acini of a Meibomian gland of subjects as compared to a subjects who do not receive the pharmaceutical composition. In some embodiments, the pharmaceutical composition, when administered to subjects suffering from meibomian gland dysfunction results in a median increase in duration of phosphorylation of Akt in meibocytes relative to subjects who do not receive the pharmaceutical composition. In some embodiments, the IGF-1 variant is a truncation. In some embodiments, the IGF-1 variant comprises or consists of the amino sequence of SEQ ID NO: 3. In some embodiments, the IGF-1 variant comprises one or more amino acid substitutions relative to wild-type IGF-1 (SEQ ID NO: 1). In some embodiments, the IGF-1 variant comprises an amino acid deletion at position 37 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises the amino sequence of SEQ ID NO: 6. In some embodiments, the IGF-1 variant comprises an amino acid substitution at position 60 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises the amino sequence of SEQ ID NO: 2. In some embodiments, the IGF-1 variant comprises an amino acid substitution at position 3 relative to wild-type IGF-1 (SEQ ID NO: 1), wherein position numbering is based on alignment of the IGF-1 variant to SEQ ID NO: 1, wherein positions are numbered from an N-terminus of SEQ ID NO: 1 to a C-terminus of SEQ ID NO: 1 starting with position 1 at the N-terminus. In some embodiments, the IGF-1 variant comprises the amino sequence of SEQ ID NO: 4. In some embodiments, the IGF-1 variant comprises the amino sequence of SEQ ID NO: 5. In some embodiments, the IGF-1 variant comprises the amino sequence of SEQ ID NO: 7. In some embodiments, the IGF-1 variant comprises the amino sequence of SEQ ID NO: 12. In some embodiments, the IGF-1 variant is coupled to a cell-penetrating peptide (CPP) or skin-penetrating peptide (SPP). In some embodiments, the IGF-1 variant is coupled to a cell-penetrating peptide selected from the group consisting of SEQ ID NO: 10 and SEQ ID NO: 11. In some embodiments, the IGF-1 variant is coupled to the skin-penetrating peptide of SEQ ID NO: 9. In some embodiments, the IGF-1 variant comprises the amino acid sequence of SEQ ID NO: 8. In some embodiments, the pharmaceutical composition comprises one or more pharmaceutically acceptable excipients. In some embodiments, the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone. In some embodiments, the at least one IGFBP comprises IGFBP2. In some embodiments, the at least one IGFBP comprises IGFBP3. In some embodiments, the at least one IGFBP comprises IGFBP1. In some embodiments, the at least one IGFBP comprises IGFBP4. In some embodiments, the at least one IGFBP comprises IGFBP5.In some embodiments, the at least one IGFBP comprises IGFBP6. In some embodiments, administration of the pharmaceutical composition to an eye or eyelid of the subject results in an increase in a size of the meibomian glands. In some embodiments, administration of the pharmaceutical composition to an eye or eyelid of the subject results in a decrease in meibomian gland atrophy. In some embodiments, administration of the pharmaceutical composition to an eye or eyelid of the subject results in a reversal of age-associated meibomian gland atrophy. In some embodiments, administration of the pharmaceutical composition to an eye or eyelid of the subject results in an increase in a function of one or more meibocytes. In some embodiments, administration of the pharmaceutical composition to an eye or eyelid of the subject results in an increase in corneal epithelial cell proliferation. In some embodiments, administration of the pharmaceutical composition to an eye or eyelid of the subject results in an increase in corneal healing. In some embodiments, administration of the pharmaceutical composition to an eye or eyelid of the subject results in an increase in IGF1 receptor (IGF1R) activation in the meibomian glands. In some embodiments, administration of the pharmaceutical composition to an eye or eyelid of the subject results in an increase duration of IGF1R activation in the meibomian glands. In some embodiments, administration of the pharmaceutical composition to an eye or eyelid of the subject results in an increase of lipid content of the meibomian glands. In some embodiments, the pharmaceutical composition, when administered to spheroids of IHGMGE cells, results in, on average, an increase in lipid content in the spheroids.

In another aspect, the present disclosure provides a kit comprising the pharmaceutical composition provided herein; and an eyedropper for delivery of the pharmaceutical composition as an eyedrop.

In another aspect, the present disclosure provides a method for treating an eye disorder in a subject in need thereof, the method comprising administering a pharmaceutical composition to a subject having an eye disorder, wherein the pharmaceutical composition comprises a therapeutically effective amount of an IGF-1 variant that has reduced affinity to an IGF binding protein (IGFBP) relative to the affinity for the interaction between wild-type IGF-1 (SEQ ID NO: 1) and the IGFBP. In some embodiments, the pharmaceutical composition is the pharmaceutical composition provided herein. the pharmaceutical composition is administered to an eye or eyelid of the subject. In some embodiments, the pharmaceutical composition is administered to the eye of the subject via an eyedropper. In some embodiments, the pharmaceutical composition is administered to an outer eyelid of the subject. In some embodiments, the pharmaceutical composition is a cream. In some embodiments, the pharmaceutical composition is administered to a subject suffering from Meibomian gland dysfunction. In some embodiments, administering the pharmaceutical composition to the subject results in an increase in surface area or volume of meibomian glands within the inner eyelid surface of the subject. In some embodiments, administering the pharmaceutical composition to the subject results in an increase in lipid content within a Meibomian gland of the subject. In some embodiments, administering the pharmaceutical composition to the subject results in an increase in release of lipid from acini of a Meibomian gland of the subject. In some embodiments, administering the pharmaceutical composition to the subject results in an increase in duration of phosphorylation of Akt in meibocytes. In some embodiments, the method does not comprise administration of any additional phospholipidosis-inducing agent. In some embodiments, the method does not comprise administration of one or both of azithromycin and doxycycline. In some embodiments, the eye disorder comprises dry eye disease. In some embodiments, the eye disorder comprises meibomian gland dysfunction. the eye disorder comprises Sjorgren's syndrome.

In another aspect, the present disclosure provides a pharmaceutical composition comprising: a therapeutically effective amount of a polypeptide comprising an amino sequence of any one of SEQ ID NOS: 1-8 or 12; and one or more pharmaceutically acceptable excipients. In some embodiments, the pharmaceutical composition is a solution for delivery as an eyedrop. In some embodiments, the pharmaceutical composition comprises a cream for administration to one or both eyelids. In some embodiments, the pharmaceutical composition is formulated for systemic delivery. In some embodiments, the polypeptide is a human IGF1R agonist. In some embodiments, administration of the pharmaceutical composition to subjects suffering from meibomian gland dysfunction results in a median increase in surface area or volume of meibomian glands within the inner eyelid surface. In some embodiments, administration of the pharmaceutical composition to subjects suffering from meibomian gland dysfunction results in a median increase in lipid content within meibomian glands. In some embodiments, administration of the pharmaceutical composition to subjects suffering from meibomian gland dysfunction results in a median increase in release of lipid from acini of the meibomian glands. In some embodiments, administration of the pharmaceutical composition to subjects suffering from meibomian gland dysfunction results in a median increase in release of lipid from acini of the meibomian glands. In some embodiments, administration of the pharmaceutical composition to subjects suffering from meibomian gland dysfunction results in a median increase in phosphorylation of Akt in meibocytes. In some embodiments, the pharmaceutical composition does not comprise any additional phospholipidosis-inducing agent. In some embodiments, the pharmaceutical composition does not comprise any one of azithromycin or doxycycline. In some embodiments, the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone. In some embodiments, the polypeptide comprises the amino sequence of SEQ ID NO: 1. In some embodiments, the polypeptide comprises the amino sequence of SEQ ID NO: 2. In some embodiments, the polypeptide comprises the amino sequence of SEQ ID NO: 3. In some embodiments, the polypeptide comprises the amino sequence of SEQ ID NO: 4. In some embodiments, the polypeptide comprises the amino sequence of SEQ ID NO: 5. In some embodiments, the polypeptide comprises the amino sequence of SEQ ID NO: 6. In some embodiments, the polypeptide comprises the amino sequence of SEQ ID NO: 7. In some embodiments, the polypeptide comprises the amino sequence of SEQ ID NO: 8. In some embodiments, the polypeptide comprises the amino sequence of SEQ ID NO: 12. In some embodiments, the polypeptide further comprises a cell-penetrating peptide (CPP) or skin-penetrating peptide (SPP). In some embodiments, the polypeptide comprises a cell-penetrating peptide selected from the group consisting of SEQ ID NO: 10 and SEQ ID NO: 11. In some embodiments, the polypeptide comprises the skin-penetrating peptide of SEQ ID NO: 9.

In another aspect, the present disclosure provides a method for treating an eye disorder in a subject in need thereof, the method comprising administering to the subject a pharmaceutical composition comprising any one of SEQ ID NOS: 1-8 or 12. In some embodiments, the pharmaceutical composition is the pharmaceutical composition provided herein. In some embodiments, the pharmaceutical composition is administered to an eye of the subject. In some embodiments, the pharmaceutical composition is administered to the eye of the subject via an eyedropper. In some embodiments, the pharmaceutical composition is administered to an outer eyelid of the subject. In some embodiments, the pharmaceutical composition is a cream. In some embodiments, administration of the pharmaceutical composition to the subject in a median increase in surface area or volume of meibomian glands on the inner eyelid surface. In some embodiments, administration of the pharmaceutical composition to the subject results in an increase in lipid content within the meibomian glands. In some embodiments, administration of the pharmaceutical composition to the subject suffering from meibomian gland dysfunction results in an increase in release of lipid from acini of the meibomian glands. In some embodiments, administration of the pharmaceutical composition to the subject results in an increase in release of lipid from acini of meibomian glands. In some embodiments, administration of the pharmaceutical composition to the subject results in an increase in phosphorylation of Akt in meibocytes. In some embodiments, the method does not comprise administration of any additional phospholipidosis-inducing agent. In some embodiments, the method does not comprise administration of any one of azithromycin or doxycycline. In some embodiments, the eye disorder comprises dry eye disease. In some embodiments, the eye disorder comprises meibomian gland dysfunction. In some embodiments, the eye disorder comprises Sjorgren's syndrome.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the dose-dependent effects of IGF-1 (SEQ ID NO: 1), IGF-1 Ea (SEQ ID NO: 7), IGF-1 Des1-3 R37X (SEQ ID NO: 6), IGF-1 Des1-3 (SEQ ID NO: 8), and IGF-1 E3R (SEQ ID NO: 4) on AKT S473 phosphorylation in DU145 cells. Wild-type IGF-1 and IGF-1 variants have similar EC₅₀ values in DU145 cells.

FIG. 2 illustrates reduced IGFBP affinity for various IGF-1 mutants. In L6 rat myoblast conditioned media containing multiple IGF binding proteins, competition assays report reduction in BP affinity for IGF-1 LR3 (SEQ ID NO: 12), LG3 (long IGF-1 E3G), long IGF-1, and IGF-1 Des 1-3 (SEQ ID NO: 3) to be 690 times, 112 times, 5.5 times, and 38 times respectively (see Francis, G L et al., 8(3) J. Mol. Endocrinol. 213-223, 1992). In a separate study using bovine IGFBP2, IGF-1 E3R (SEQ ID NO: 4) and IGF E3G were reported to have reduction in binding affinity for IGFBP by 230 times and 59 times, respectively (see King, R. et al., 8 J. Mol. Endocrinol. 29-41, 1992).

FIGS. 3A and 3B provide that IGF-1 LR3 (SEQ ID NO: 12) and IGF-1 E3R (SEQ ID NO: 4) are less inhibited by IGFBP2 and IGFBP3 compared to wild-type IGF-1 (SEQ ID NO: 1). FIG. 3A depicts the effects of wild-type IGF-1, IGF-1 LR3, and IGF-1 E3R on AKT S473phosphorylation in DU145 cells in the presence of IGFBP2 at an IGF-1 to IGFBP2 ratio of 1:1, 1:2,or 1:4, respectively. FIG. 3B depicts the effects of wild-type IGF-1, IGF-1 LR3, and IGF-1 E3R on AKT S473 phosphorylation in DU145 cells in the presence of IGFBP3 at an IGF-1 to IGFBP3 ratio of 1:1, 1:2, or 1:4, respectively.

FIG. 4 depicts the dose-dependent effects of IGF-1 on AKT S473 phosphorylation in immortalized human meibomian gland epithelial cells (IHMGECs). The EC₅₀ value for IGF-1 is about 0.07 nanomolar (nM).

FIG. 5 depicts the results of live cell imaging assays: IHMGECs spread out and proliferate at increased rates upon IGF-1 stimulation in a dose-dependent manner.

FIG. 6 depicts the results of human IGF-1 stimulation in IGHMECs following the cell viability assay: the cells proliferate in response to IGF-1 in a dose-dependent matter.

FIGS. 7A and 7B depict immortalized Human Meibomian Gland Epithelial (IHMGE) cells form spheroids resembling meibomian gland acini when grown in a 3D culture. FIG. 7A depicts images of 2D and 3D spheroid cultures; brightfield images were collected with transmitted light. FIG. 7B depicts immunofluorescence images of nuclei markers, spheroids expressing markers (Krt5) of the meibomian gland acini basal compartment, and the merged images of the two.

FIG. 8 provides that spheroids grown in the presence of IGF-1 E3R are significantly larger than those grown with wild-type IGF-1. The diameters of spheroids cultured in the presence of 0 nM, 0.1 nM, 1.6 nM, or 10 nM of IGF-1 or IGF-1 E3R were measured on day seven of differentiation.

FIG. 9 provides IGF-1 and IGF-1 E3R proportionally increase lipid content proportional to spheroid diameter. Lipidtox intensity of spheroids cultured in the presence of 0 nM, 0.1 nM, 1.6 nM, or 10 nM of IGF-1 or IGF-1 E3R were measured on day seven of differentiation.

FIG. 10 provides that IGFBP2 is expressed higher compared to IGFBP1, IGFBP3, IGFBP4 and IGFBP6 in IHMGE spheroid cultures.

FIG. 11A illustrates the study design of transcriptional analysis of the effects of wild-type IGF1 treatment on IHMGE spheroids. FIG. 11B depicts that four IGFBPs were significantly upregulated by treatment of IHMGE spheroids with wild-type IGF-1 in the transcriptional analysis. FIG. 11C depicts that IGF-1 treatment significantly upregulates genes involved in fatty acid transport, lipid synthesis, and meibogenesis.

FIG. 12 provides that IGFBP2 is highly expressed in the basal compartment of the meibomian gland in mice. FIG. 12 depicts representative immunofluorescent images of meibomian glands labeling both of nuclei (DAPI, green) and proliferating cells (Ki67, red), IGFBP2, and the merged image of the two.

FIGS. 13A and 13B depict increased proliferation in meibomian gland acini in young mice compared to older mice. Cell proliferation in the Meibomian gland is reduced with age. FIG. 13A depicts that the number of proliferating cells (Ki67+) in acini of aged meibomian glands was lower than that observed in young ones. Statistical comparison made using student's t-test, n=10 per group. FIG. 13B depicts representative immunofluorescent images of young (above) and old (below) meibomian glands labeling nuclei (DAPI, green) and proliferating cells (Ki67, red). Cells positive for both markers (Double positive) in acini are highlighted in blue using image analysis in ImageJ.

FIGS. 14A and 14B provide a graph and images showing increased proliferation in meibomian gland acini in aged mice upon systemic IGF-1 LR3 treatment. FIG. 14A shows that IGF1 LR3 can induce proliferation in the meibomian gland of aged mice. Mice were administered 2 doses of IGF-1 LR3 at 10 mg/kg by intraperitoneal injection 12 hours apart and taken down at 24 or 48 hours after the first dose. The number of proliferating cells (Ki67+) in acini of vehicle-treated (left) or IGF-1 LR3-treated (right) meibomian glands was quantified by comparing the number of Ki67 positive cells in meibomian gland acini. Statistical comparison made using Two-way ANOVA followed by Tukey's multiple comparison test. N=10 per group. FIG. 14B shows representative immunofluorescent images of meibomian glands from 24 hour vehicle-treated (above) and 24 hour IGF-1 LR3-treated (below) labeling nuclei (DAPI, green) and proliferating cells (Ki67, red). Cells positive for both markers (Double positive) in acini are highlighted in blue using image analysis in ImageJ.

FIGS. 15A and 15B provide a graph and images showing atrophy reversal and increased size in meibomian gland area in aged mice upon systemic IGF-1 LR3 treatment. FIG. 15A depicts that mice were untreated (young) or administered a IGF-1 LR3 at 10 mg/kg by intraperitoneal injection on 5 consecutive days per week for 4 weeks and subjected to transillumination meibography before sacrifice. Quantitation of meibography data shows increased gland area in IGF-1 LR3-treated old mice compared to vehicle-treated control old mice. Pairwise comparisons were made using one-tailed Mann-Whitney test, n=7-10 mice/group. FIG. 15B depicts representative images from transillumination meibography experiments with one acinus area in each image.

FIGS. 16A and 16B provide a graph and images showing increased lipid synthesis in Meibomian glands regardless of age upon systemic IGF-1 LR3 treatment. FIG. 16A depicts that mice were untreated (young) or administered a IGF-1 LR3 at 10 mg/kg by intraperitoneal injection on 5 consecutive days per week for 4 weeks before sacrifice. Nuclei (DAPI, red) and meibomian gland lipids (Lipidtox, green) were labeled in eyelid sections and lipid droplet density was quantified in ImageJ. Statistical comparisons were made using One-way ANOVA followed by Tukey's multiple comparison test. N=7-10 per group. FIG. 16B depicts representative images from each group.

FIG. 17 provides a graph showing that IGF-1 LR3 can be delivered systemically or by ocular drops to activate IGF1 receptor in the eyelid. Mice were administered a single dose of IGF-1 LR3 at 10 mg/kg by intraperitoneal injection or treated with vehicle (PBS) or PBS containing 5 mg/ml IGF-1 LR3 via eyedrops administered to the ocular surface. Animals were sacrificed 30 minutes after dosing and eyelids were homogenized and assays for pAKT levels by pAKT ELISA as a reporter for activation of IGF1R. Statistical comparisons were made using One-way ANOVA followed by Tukey's multiple comparison test. n=3 per group.

FIG. 18 provides that IGF-1 LR3 prolongs duration of increased IGF1R signaling compared to wild-type IGF-1. The effects of wild-type IGF-1 and IGF-1 LR3 on AKT S473 phosphorylation was measured after 0.5 hour or 2 hours post dosing. Statistical analysis: One-way Anova. n=3 per group.

FIG. 19 provides that both IGF-1 LR3 and IGF-1 Des 1-3, which are reduced in their ability to bind binding proteins, prolong duration of IGF1R activation at 2 hours post dosing compared to wild-type IGF-1. n=5/group. The effects of IGF-1, IGF-1 LR3 and IGF-1 Des 1-3 on AKT S473 phosphorylation was measured at 2 hours post dosing in eyelids of tested animals. Statistical analysis: One-way Anova.

FIG. 20 provides that IGF-1 LR3 and IGF-1 E3R, which are reduced in their ability to bind binding proteins, are similarly potent in vivo compared to IGF-1 at 1 hour post dosing. The effects of IGF-1, IGF-1 LR3 and IGF-1 E3R on AKT S473 phosphorylation was measured at 1 hour post dosing in eyelids of tested animals. n=3/group. Statistical analysis: One-way Anova.

FIG. 21 provides that IGF-1 LR3 stimulates dose-responsive proliferation in the aged mouse meibomian glands. The number of Ki67-labeled proliferating cells per 100 μm perimeter was measured following the treatment of 0 mg/ml, 0.3 mg/ml, 1 mg/ml, or 3 mg/ml of IGF-1 LR3 eyedrop in young or aged mice. n=5/group. Statistical analysis: One-way Anova.

FIGS. 22A and 22B provide that IGF-1 LR3 regenerates atrophied meibomian glands in aged mice. Daily treatment with IGF-1 LR3 for 1 month increases area of meibomian glands comparing pre-and post-treatment. FIG. 22A depicts individual animal glands before and after treatment of IGF-1 LR3 or vehicle treatment. FIG. 22B depicts the quantification of changes in gland area with the treatment of IGF-1 LR3 or vehicle as assessed by transillumination meibography. n=10/group. Statistical analysis: One-way ANOVA.

FIG. 23 provides that IGF-1 LR3 and IGF-1 E3R induce similar levels of proliferation in meibomian glands in aged mice after two weeks of daily eyedrop dosing. The number of Ki67-labeled proliferating cells per 100 μm perimeter was measured following the treatment of vehicle or IGF-1 ER3 eyedrop in young or aged mice. n=4-5/group. Statistical analysis: One-way ANOVA.

### DETAILED DESCRIPTION OF THE INVENTION

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” means plus or minus 10% of the number that the term refers to.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable” refers to approved or approvable by a regulatory agency of the Federal or a state government or listed in the U.S. Pharmacopeia (U.S.P.) or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not. The term “sequence identity” or a percent (%) of sequence identity, as used herein is the percentage of residues in a candidate sequence that are identical with the residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried. An effective amount of an active agent may be administered in a single dose or in multiple doses.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

While certain embodiments of the present application have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

### Polypeptides

Meibomian glands comprise meibocytes, cells that form clusters in acini. When functioning normally, meibocytes may proliferate, differentiate, and produce meibum—a lipid-rich secretion. The dysfunction of Meibomian glands can be a catalyst in the onset of dry eye disease. In one exemplary mechanism, a decrease in insulin-like growth factor (IGF-1) binding to IGF-1 receptor (IGF1R) and resulting decrease in IGF1R activation (or other deficient signaling of IGF1R), may affect the morphology and function of Meibomian glands. For example, it may lead to Meibomian gland atrophy and dysfunction, or dry eye disease.

The present disclosure provides polypeptides and methods of treatment for Meibomian glands or dry eye disease. The polypeptides may modulate (e.g., upregulate or otherwise activate) the activity of the IGF1R. The polypeptide may have reduced affinity to at least one IGF binding protein (IGFBP) relative to wild-type IGF-1 (SEQ ID NO: 1) to the IGFBP. In one aspect, the polypeptides act locally. The level of one or both of (1) free endogenous IGF-1 or (2) the polypeptides (IGF-1 variants with reduced affinity to one or more IGFBPs) that bind to IGF1R may be increased upon administration to a subject. The local activation of IGF1R by the polypeptides may result in a prolonged or extended pharmacodynamic effect. In another aspect, the polypeptides act systemically. The systemic half-life of the polypeptides decreases due to their reduced affinity to IGFBPs.

IGF-1 and IGF-1 variants can bind to one or more IGFBPs. IGFBPs can lengthen the half-life of circulating wild-type IGF-1. In one aspect, IGFBPs may act to enhance systemic IGF-1 signaling due to increased levels of circulating IGF-1 and IGF-1 variants. In another aspect, IGFBPs may act to attenuate local IGF-1 signaling due to decreased levels of locally free IGF-1 and IGF-1 variants. In one aspect, the one or more IGFBPs may include one or more of be IGFBP1, IGFBP2, IGFBP3, IGFBP4, IGFBP5, IGFBP6, or any combination thereof. In some embodiments, the one or more IGFBPs comprise IGFBP1. In some embodiments, the one or more IGFBPs comprise IGFBP2. In some embodiments, the one or more IGFBPs comprise IGFBP3. In some embodiments, the one or more IGFBPs comprise IGFBP4. In some embodiments, the one or more IGFBPs comprise IGFBP1 and IGFBP2. In some embodiments, the one or more IGFBPs comprise IGFBP1 and IGFBP3. In some embodiments, the one or more IGFBPs comprise IGFBP1 and IGFBP4. In some embodiments, the one or more IGFBPs comprise IGFBP2 and IGFBP3. In some embodiments, the one or more IGFBPs comprise IGFBP2 and IGFBP3. In some embodiments, the one or more IGFBPs comprise IGFBP3 and IGFBP4. In some embodiments, the one or more IGFBPs comprise IGFBP1, IGFBP2, and IGFBP3. In some embodiments, the one or more IGFBPs comprise IGFBP1, IGFBP2, and IGFBP4. In some embodiments, the one or more IGFBPs comprise IGFBP1, IGFBP3, and IGFBP4.In some embodiments, the one or more IGFBPs comprise IGFBP2, IGFBP3, and IGFBP4. In some embodiments, the one or more IGFBPs comprise IGFBP1, IGFBP2, IGFBP3, and IGFBP4.

TABLE 1
Sequences of polypeptides for treating Meibomian
glands or dry eye disease

| SEQ ID | | |
|---|---|---|
| NO: | Name | Sequence |
| 1 | Wild-type | GPETLCGAELVDALQFVCGDRGFYFNKPTGYGSSSRRA |
| | IGF-1 | PQTGIVDECCFRSCDLRRLEMYCAPLKPAKSA |
| 2 | IGF-1 Y60L | GPETLCGAELVDALQFVCGDRGFYFNKPTGYGSSSRRA |
| | | PQTGIVDECCFRSCDLRRLEMLCAPLKPAKSA |
| 3 | IGF1 Des1-3 | TLCGAELVDALQFVCGDRGFYFNKPTGYGSSSRRAPQT |
| | | GIVDECCFRSCDLRRLEMYCAPLKPAKSA |
| 4 | IGF1 E3R | GPRTLCGAELVDALQFVCGDRGFYFNKPTGYGSSSRRA |
| | | PQTGIVDECCFRSCDLRRLEMYCAPLKPAKSA |
| 5 | IGF1 R37X | GPETLCGAELVDALQFVCGDRGFYFNKPTGYGSSSRAP |
| | | QTGIVDECCFRSCDLRRLEMYCAPLKPAKSA |
| 6 | Des1-3 | TLCGAELVDALQFVCGDRGFYFNKPTGYGSSSRAPQTG |
| | R37X | IVDECCFRSCDLRRLEMYCAPLKPAKSA |
| 7 | IGF1Ea | GPETLCGAELVDALQFVCGDRGFYFNKPTGYGSSSRRA |
| | | PQTGIVDECCFRSCDLRRLEMYCAPLKPAKSARSVRAQ |
| | | RHTDMPKTQKEVHLKNASRGSAGNKNYRM |
| 8 | CPP-Des1-3 | MRAAAPAVAATLCGAELVDALQFVCGDRGFYFNKPTG |
| | | YGSSSRRAPQTGIVDECCFRSCDLRRLEMYCAPLKPAKS |
| | | A |
| 9 | skin- | MRAAAPAVAA |
| | penetrating | |
| | peptide | |
| 10 | CPP | CGRKKRRQRRRPPQC |
| 11 | CPP | RRRRRRRRR |
| 12 | IGF-1 LR3 | MFPAMPLSSLFVNGPRTLCGAELVDALQFVCGDRGFYF |
| | | NKPTGYGSSSRRAPQTGIVDECCFRSCDLRRLEMYCAPL |
| | | KPAKSA |

In some embodiments, the polypeptide comprises or consists of SEQ ID NO: 1 (native human IGF-1). The polypeptide may comprise or consist of 70 amino acids. The polypeptide may have potent binding to IGF1R. The polypeptide may have potent binding to the one or more IGFBPs. An affinity of native IGF-1 for IGF1R may be in a range of about 1 nanomolar (nM) to about 10 nM, as measured by an affinity assay.

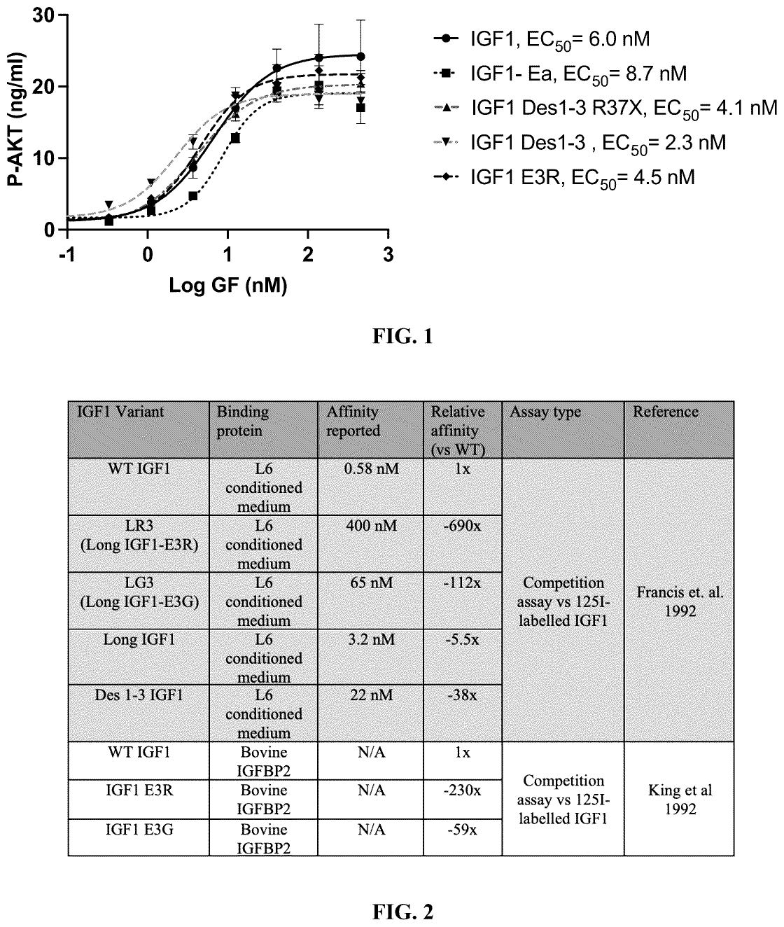

In one aspect, the polypeptide is an IGF-1 variant. In some embodiments, the IGF-1 variant has at least 60% sequence identity to SEQ ID NO: 1. In some embodiments, the IGF-1 variant has at least 65% sequence identity to SEQ ID NO: 1. In some embodiments, the IGF-1 variant has at least 70% sequence identity to SEQ ID NO: 1. In some embodiments, the IGF-1 variant has at least 75% sequence identity to SEQ ID NO: 1. In some embodiments, the IGF-1 variant has at least 80% sequence identity to SEQ ID NO: 1. In some embodiments, the IGF-1 variant has at least 85% sequence identity to SEQ ID NO: 1. In some embodiments, the IGF-1 variant has at least 90% sequence identity to SEQ ID NO: 1. In some embodiments, the IGF-1 variant has at least 91% sequence identity to SEQ ID NO: 1. In some embodiments, the IGF-1 variant has at least 92% sequence identity to SEQ ID NO: 1. In some embodiments, the IGF-1 variant has at least 93% sequence identity to SEQ ID NO: 1. In some embodiments, the IGF-1 variant has at least 94% sequence identity to SEQ ID NO: 1. In some embodiments, the IGF-1 variant has at least 95% sequence identity to SEQ ID NO: 1. In some embodiments, the IGF-1 variant has at least 96% sequence identity to SEQ ID NO: 1. In some embodiments, the IGF-1 variant has at least 97% sequence identity to SEQ ID NO: 1. In some embodiments, the IGF-1 variant has at least 98% sequence identity to SEQ ID NO: 1. In some embodiments, an affinity of the IGF-1 variant for IGF1R may be in a range of about 1 nanomolar (nM) to about 10 nM, as measured by an affinity assay. In some embodiments, the polypeptide (e.g., IGF-1 variant) has an EC50 of greater than 1 nM, greater than 2 nM, greater than 3 nM, greater than 4 nM, or greater than 5 nM as determined via the assay described in connection with FIG. 1. In some embodiments, the EC50 of the polypeptide is between 1 nM and 20 nM, between 1 nM and 15 nM, between 1 nM and 10 nM, between 2 nM and 10 nM, or between 3 nM and 5 nM. In some embodiments, the polypeptide (e.g., IGF-1 variant) does not bind or binds very weakly to the insulin receptor. For instance, in some cases, the Kd of the interaction of the polypeptide (e.g., IGF-1 variant) is >10-fold weaker than the binding of insulin to the insulin receptor.

In some embodiments, the IGF-1 variant comprises a truncation of the amino acid sequence of SEQ ID NO: 1. In some embodiments, the truncation is at the N-terminus of SEQ ID NO: 1. In some embodiments, the truncation is at the C-terminus of SEQ ID NO: 1. In some embodiments, the IGF-1 variant comprises a truncation that includes a deletion of at least about 1 amino acid. In some embodiments, the IGF-1 variant comprises a truncation that includes a deletion of at least about 2 amino acids. In some embodiments, the IGF-1 variant comprises a truncation that includes a deletion of at least about 3 amino acids. In some embodiments, the IGF-1 variant comprises a truncation that includes a deletion of at least about 4 amino acids. In some embodiments, the IGF-1 variant comprises a truncation that includes a deletion of at least about 5 amino acids. In some embodiments, the IGF-1 variant comprises a truncation that includes a deletion of at least about 6 amino acids. In some embodiments, the IGF-1 variant comprises a truncation that includes a deletion of at least about 7 amino acids. In some embodiments, the IGF-1 variant comprises a truncation that includes a deletion of at least about 8 amino acids. In some embodiments, the IGF-1 variant comprises a truncation that includes a deletion of at least about 9 amino acids. In some embodiments, the IGF-1 variant comprises a truncation that includes a deletion of at least about 10 amino acids. In some embodiments, the IGF-1 variant comprises a truncation that includes a deletion of at least about 11 amino acids. In some embodiments, the IGF-1 variant comprises a truncation that includes a deletion of at least about 12 amino acids.

In some embodiments, the IGF-1 variant is an extension of the amino acid sequence of SEQ ID NO: 1. In some embodiments, the extension is at the C-terminus of SEQ ID NO: 1. In some embodiments, the extension is at the N-terminus of SEQ ID NO: 1. In some embodiments, the extension has at least about 2 amino acids. In some embodiments, the extension has at least about 3 amino acids. In some embodiments, the extension has at least about 4 amino acids. In some embodiments, the extension has at least about 5 amino acids. In some embodiments, the extension has at least about 6 amino acids. In some embodiments, the extension has at least about 7 amino acids. In some embodiments, the extension has at least about 8 amino acids. In some embodiments, the extension has at least about 9 amino acids. In some embodiments, the extension has at least about 10 amino acids. In some embodiments, the extension has at least about 15 amino acids. In some embodiments, the extension has at least about 20 amino acids. In some embodiments, the extension has at least about 35 amino acids. In some embodiments, the extension has at least about 40 amino acids. In some embodiments, the extension has at least about 45 amino acids. In some embodiments, the extension has at least about 50 amino acids.

In some embodiments, the IGF-1 variant comprises one or more amino acid substitutions relative to wild-type IGF-1 (SEQ ID NO: 1). In some embodiments, the IGF-1 variant comprises an amino acid substitution at position 3 relative to wild-type IGF-1 (SEQ ID NO: 1). In some embodiments, the amino acid substitution at position 3 is an arginine. In some embodiments, the IGF-1 variant comprises an amino acid substitution at position 60 relative to wild-type IGF-1 (SEQ ID NO: 1). In some embodiments, the amino acid substitution at position 60 is a leucine. Position numbering can be based on alignment of the IGF-1 variant to SEQ ID NO: 1, with positions numbered from an N-terminus of SEQ ID NO: 1 to a C-terminus of SEQ ID NO: 1 starting with position 1 at the N-terminus of SEQ ID NO: 1. In some embodiments, the polypeptide comprises IGF-1 Y60L. In some embodiments, the polypeptide comprises IGF-1 E3R. In some embodiments, the polypeptide comprise a native IGF-1 with a deletion at R37.

In some embodiments, the IGF-1 variant comprises a truncation of the amino acid sequence of SEQ ID NO: 1 and one or more amino acid substitutions relative to wild-type IGF-1 (SEQ ID NO: 1). In some embodiments, the IGF-1 variant comprises a truncation of 3 amino acids at the N-terminus of SEQ ID NO: 1 and an amino acid deletion at position 37 relative to wild-type IGF-1 (SEQ ID NO: 1). In some embodiments, the IGF-1 variant comprises a deletion relative to SEQ ID NO: 1. In some embodiments, the IGF-1 variant comprises an amino acid deletion at position 37 relative to wild-type IGF-1 (SEQ ID NO: 1).

In some embodiments, the IGF-1 variant comprises the amino sequence of SEQ ID NO: 2. In some embodiments, the IGF-1 variant comprises the amino sequence of SEQ ID NO: 3. In some embodiments, the IGF-1 variant comprises the amino sequence of SEQ ID NO: 4. In some embodiments, the IGF-1 variant comprises the amino sequence of SEQ ID NO: 5. In some embodiments, the IGF-1 variant comprises the amino sequence of SEQ ID NO: 6. In some embodiments, the IGF-1 variant comprises the amino sequence of SEQ ID NO: 7. In some embodiments, the IGF-1 variant comprises the amino sequence of SEQ ID NO: 8. In some embodiments, the IGF-1 variant comprises the amino sequence of SEQ ID NO: 12.

The polypeptide may comprise or consist of about 66 amino acids. The polypeptide may comprise or consist of about 67 amino acids. The polypeptide may comprise or consist of about 69 amino acids. The polypeptide may comprise or consist of about 150 amino acids. The polypeptide may comprise or consist of about 70 amino acids. The polypeptide may comprise or consist of about 83 amino acids. The polypeptide may comprise or consist of about 105 amino acids.

The polypeptide may have a decreased systemic and/or local half-life upon administration to a healthy adult compared to native human IGF-1. In some embodiments, the systemic and/or local half-life of the polypeptide upon administration to a healthy adult decreases at least 5% compared to native human IGF-1. In some embodiments, the systemic and/or local half-life of the polypeptide upon administration to a healthy adult decreases at least 10% compared to native human IGF-1. In some embodiments, the systemic and/or local half-life of the polypeptide upon administration to a healthy adult decreases at least 15% compared to native human IGF-1. In some embodiments, the systemic and/or local half-life of the polypeptide upon administration to a healthy adult decreases at least 20% compared to native human IGF-1. In some embodiments, the systemic and/or local half-life of the polypeptide upon administration to a healthy adult decreases at least 30% compared to native human IGF-1. In some embodiments, the systemic and/or local half-life of the polypeptide upon administration to a healthy adult decreases at least 40% compared to native human IGF-1. In some embodiments, the systemic and/or local half-life of the polypeptide upon administration to a healthy adult decreases at least 50% compared to native human IGF-1.

The polypeptide may have reduced affinity to at least one IGFBP relative to native human IGF-1 (SEQ ID NO: 1) to the IGFBP. In some embodiments, the polypeptide has reduced affinity to IGFBP1 relative to native human IGF-1 (SEQ ID NO: 1). In some embodiments, the polypeptide has reduced affinity to IGFBP2 relative to native human IGF-1 (SEQ ID NO: 1). In some embodiments, the polypeptide has reduced affinity to IGFBP3 relative to native human IGF-1 (SEQ ID NO: 1). In some embodiments, the polypeptide has reduced affinity to IGFBP4 relative to native human IGF-1 (SEQ ID NO: 1). In some embodiments, the polypeptide has reduced affinity to IGFBP5 relative to native human IGF-1 (SEQ ID NO: 1). In some embodiments, the polypeptide has reduced affinity to IGFBP6 relative to native human IGF-1 (SEQ ID NO: 1). In some embodiments, the polypeptide has reduced affinity to IGFBP3 and IGFBP2 relative to native human IGF-1 (SEQ ID NO: 1).

In some embodiments, the polypeptide may further comprise a 13 amino acid sequence (MFPAMPLLSLFVN (SEQ ID NO: 13)) at its C-terminal or N-terminal sequence.

IGFBPs play a key role in extending the systemic half-life of Insulin-like Growth Factor 1 (IGF-1) in the human body. While the unbound form of wild-type IGF-1 has a systemic half-life of 10 to 20 minutes, the binding of IGF-1 to IGFBPs increases its half-life to several hours. This extended half-life is generally thought to increase the duration of IGF-1's biological effect. This is most clearly seen in Laron Syndrome, a form of dwarfism in which patients are unable to make, among other things, IGFBPs. Laron dwarfs given high doses of systemic IGF1 do not grow very much, in part because IGF-1 cannot persist in circulation without the dramatic half-life extension provided by IGFBP binding.

It is therefore surprising that, as described herein, when IGF1 variants that evade IGFBPs are locally administered (e.g., as an eye drop to treat meibomian gland dysfunction), that the opposite phenomenon is observed. For instance, the desired pharmacodynamic effects of wild-type IGF-1 treatment are seen at lower levels and across shorter time frames relative to IGF-1 variants (e.g., IGF1 LR3, IGF des 1-3, IGF E3R) that possess the ability to evade one or more IGFBPs. Stated differently, unexpectedly, relative to wild-type IGF-1 (SEQ ID NO: 1), variants that evade one or more IGF-1 binding proteins (e.g., IGFBP2) can exhibit extended and/or improved pharmacodynamic effects when administered locally, even though binding to one or more binding proteins has been understood as being important to extending half-life and function of IGF-1.

### Cell-Penetrating Peptides (CPPs) and Skin-Penetrating Peptides (SPPs)

Polypeptides as disclosed herein may further comprise a cell-penetrating peptide (CPP) or a skin-penetrating peptide (SPP). The stratum corneum of skin generally comprises keratin-enriched dead cells floating in layered, lipid domains, and this functions as a barrier to the environment. This structure may inhibit absorption and transport of macromolecules to the dermis and beyond. CPPs or SPPs as disclosed herein may have high transduction efficiency, thereby enabling transdermal delivery. CPPs or SPPs as disclosed herein may modify the structure of a skin barrier to allow molecules that they are co-formulated with (even if not conjugated to) to enter and/or translocate across the skin.

The IGF-1 variant can be coupled to a cell-penetrating peptide (CPP) or skin-penetrating peptide (SPP) described herein. A CPP or SPP may comprise any one of SEQ ID NOS: 9-11. In some embodiments, a CPP or SPP comprises SEQ ID NO: 9. In some embodiments, a CPP or SPP comprises SEQ ID NO: 10. In some embodiments, a CPP or SPP comprises SEQ ID NO: 11. In some embodiments, a polypeptide comprising any one of SEQ ID NOS: 1-8, 12, or any combination thereof is coupled to a CPP or SPP. In some embodiments, a polypeptide comprising SEQ ID NO: 1 is coupled to a CPP or SPP. In some embodiments, a polypeptide comprising SEQ ID NO: 2 is coupled to a CPP or SPP. In some embodiments, a polypeptide comprising SEQ ID NO: 3 is coupled to a CPP or SPP. In some embodiments, a polypeptide comprising SEQ ID NO: 4 is coupled to a CPP or SPP. In some embodiments, a polypeptide comprising SEQ ID NO: 5 is coupled to a CPP or SPP. In some embodiments, a polypeptide comprising SEQ ID NO: 6 is coupled to a CPP or SPP. In some embodiments, a polypeptide comprising SEQ ID NO: 7 is coupled to a CPP or SPP. In some embodiments, a polypeptide comprising SEQ ID NO: 8 is coupled to a CPP or SPP. In some embodiments, a polypeptide comprising SEQ ID NO: 12 is coupled to a CPP or SPP.

Compositions or formulations delivered transdermally may be configured to better reach steady state level as compared to compositions or formulations not delivered by transdermal delivery. Compositions or formulations delivered transdermally may also be capable of bypassing hepatic metabolism, and reduce unwanted systemic side effects, thereby increasing patient compliance. CPPs or SPPs as disclosed herein may comprise or consist of about 5 to about 30 amino acids. CPPs or SPPs as disclosed herein may enable a polypeptide to penetrate skin on an eyelid, thereby enabling the polypeptide to become absorbed into the eyelid. SPPs as disclosed herein may comprise or consist of a hydrophobic peptide. CPPs as disclosed herein comprise or consist of a cationic peptide. The cationic peptide may have one or more charged amino acids, e.g., arginine. The CPP or SPP may present low cytotoxicity to cells, e.g., human cells. SPPs as disclosed herein may comprise a macromolecule transduction domain (MTD). The MTD may comprise or consist of MRAAAPAVAA (SEQ ID NO: 9). The MTD may be derived from a membrane translocation sequence (MTS) of a Kaposi fibroblast growth factor (FGF-4) signal peptide. CPPs as disclosed herein may comprise or consist of SEQ ID NO: 10. The polypeptide of SEQ ID NO: 10 may be penetrate the epidermis and dermis of skin, and may be useful in treating antioxidant disorders. The polypeptide of SEQ ID NO: 10 may be delivered via, e.g., a skin spray. CPPs as disclosed herein may comprise or consist of SEQ ID NO: 11. The polypeptide of SEQ ID NO: 11 may be penetrate the epidermis and dermis of skin, and may be useful in treating antioxidant disorders. The polypeptide of SEQ ID NO: 11 may be delivered via, e.g., a skin spray.

### Pharmaceutical Compositions

The present disclosure provides pharmaceutical compositions for treating Meibomian glands or dry eye disease. The pharmaceutical composition may be applied topically, e.g., as eye drops or as a cream. The pharmaceutical composition may be applied to an eyelid. The pharmaceutical composition may be formulated for systemic delivery. The pharmaceutical composition may penetrate an eyelid.

The pharmaceutical composition may comprise a pharmaceutically acceptable carrier or adjuvant, such as, for example, a hyaluronate (or hyaluronic acid), an electrolyte, an ophthalmic demulcent, an excipient, an astringent, a vasoconstrictor and/or an emollient. Examples of pharmaceutically acceptable excipients may include the one or more pharmaceutically acceptable excipients comprises one or more of water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, and polyvinylpyrrolidone. Examples of electrolytes may include sodium chloride, potassium chloride, sodium bicarbonate, potassium bicarbonate, calcium chloride, magnesium chloride, trisodium citrate, hydrochloric acid, sodium hydroxide, and mixtures thereof. Pharmaceutical compositions as disclosed herein may comprise a solution having one or more electrolytes. For instance, in some embodiments, the electrolyte-containing solution comprises one or more of sodium chloride, potassium chloride, sodium bicarbonate, potassium bicarbonate, calcium chloride, magnesium chloride, trisodium citrate, hydrochloric acid, or sodium hydroxide. In some instances, the mole percent of sodium chloride is from about 40% to about 60%. In some instances, the mole percent of sodium chloride is about 40%, 45%, 50%, 55%, or 60%. In some instances, the mole percent of potassium chloride is about 1% to about 20%. In some instances, the mole percent of potassium chloride is about 1%, 2%, 3%, 4%, 5%, 10%, 15%, or 20%. In some instances, the mole percent of sodium bicarbonate is from about 1% to about 25%. In some instances, the mole percent of sodium bicarbonate is about 1%, 2%, 3%, 4%, 5%, 10%, 15%, 20%, or 25%. In some instances, the mole percent of potassium bicarbonate is about 0% to about 10%. In some instances, the mole percent of potassium bicarbonate is about 0%, 1%, 2%, 3%, 4%, 5%, 6%, 7%, 8%, 9%, or 10%. In some instances, the mole percent of calcium chloride is about 0% to 10% of. In some instances, the mole percent of calcium chloride is about 0%, 1%, 2%, 3%, 4%, 5%, 6%, 7%, 8%, 9%, or 10%. In some instances, the mole percent of magnesium chloride is about 0% to 10% of. In some instances, the mole percent of magnesium chloride is about 0%, 1%, 2%, 3%, 4%, 5%, 6%, 7%, 8%, 9%, or 10%. In some instances, the mole percent of trisodium citrate is about 0% to 10% of. In some instances, the mole percent of trisodium citrate is about 0%, 1%, 2%, 3%, 4%, 5%, 6%, 7%, 8%, 9%, or 10%. In some instances, the mole percent of hydrochloric acid is about 0% to about 30%. In some instances, the mole percent of hydrochloric acid is about 0%, 1%, 2%, 3%, 4%, 5%, 6%, 7%, 8%, 9%, or 10%, 15%, 20%, 25%, or 30%. In some instances, the mole percent of sodium hydroxide about 0% to about 30%. In some instances, the mole percent of sodium hydroxide about 0%, 1%, 2%, 3%, 4%, 5%, 6%, 7%, 8%, 9%, or 10%, 15%, 20%, 25%, or 30%.

In some embodiments, excipients may include one or more permeation enhancers. The term “permeation enhancer” or “penetration enhancer” may refer to a compound or mixture of compounds that interact with one or more skin constituents to promote drug penetration into and/or through the skin (e.g., through skin on an outer eyelid to one or more Meibomian glands). Any suitable permeation enhancer known in the art may be used in the pharmaceutical compositions described herein, including, for example, surfactants (e.g., ionic (anionic, cationic, zwitterionic) surfactants (such as sodium lauryl sulfate, sodium laureate, etc.) non-ionic surfactants (such as Tween-80, other polysorbates, etc.), and any combinations thereof), bile salts and derivatives thereof (e.g, sodium glyacolate, sodium deoxycholate, etc.), fatty acids and derivatives thereof (e.g, oleic acid, caprylic acid, esters of fatty acids such as isopropyl myrisate, etc.), chelating agents (e.g, EDTA, citric acid, etc.), sulphoxides (e.g, DMSO, DMA, DMF, etc.), polyols (e.g, diethylene glycol monoethyl ether, PG, polyethylene glycols (PEGs), glycerol, polyglycols, etc.), alcohols (e.g, alkanols, alkenols, glycols, etc.), hydrocarbons (e.g, alkanes, alkenes, halogenated alkanes, squalene, squalene, mineral oil, etc.), amines, amides (e.g, cyclic amides, acyclic amides, azones, pyrrolidones, urea and derivatives thereof, etc.), others (e.g, terpenes and terpenoids, essential oils (such as eucalyptus oil, peppermint oil, turpentine oil, etc.), phospholipids, cyclic oligosaccharides (such as cyclodextrins), amino acids and thioacyl derivatives of amino acids, alkyl amino esters and oxazolidinones, enzymes, ketones (such as macrocyclic ketones), etc.), hyaluronic acid, benzalkonium chloride, and any combinations thereof.

In some embodiments, the pharmaceutically acceptable excipient(s) s are adapted for transdermal delivery through an outer eyelid by formulating the excipient(s) to 1) achieve improved spreadability on the outer eyelid surface; and/or 2) avoid flow from the outer eyelid surface onto the corneal surface. In some embodiments, the pharmaceutically acceptable excipient(s) comprise characteristics and rheological features of improved spreadability, resulting in easier administration and spreading onto the eyelid surface, and absence of flow at the body temperature of a subject, particularly after being applied to the skin of a subject. In some embodiments, pharmaceutical compositions of the present disclosure are formulated such that the cohesiveness of the formulation does not significantly change after application to the skin (e.g., eyelids) of a subject. Examples of suitable additives that convey a suitable cohesiveness of the formulation may include, for example, additives that increase viscosity of the formulation such as waxes, paraffins, and elastomers. In some embodiments, the viscosity of the formulation does not significantly change when heated from room temperature to a temperature closer to the body temperature of a subject.

In some embodiments, the pharmaceutically acceptable formulation is an ointment comprising a water-miscible ointment base. In some embodiments, the pharmaceutically acceptable formulation is an ointment comprising a paraffinic ointment base. In some embodiments, the ointment comprises one or more of white soft paraffin, mineral oil, propylene glycol, ST cyclomethicone-5NF, labrasol, propylene carbonate, steareth 2, ST emulsifier 10, and ST elastomer-10. In some embodiments, the ointment comprises white soft paraffin, mineral oil, propylene glycol, ST cyclomethicone-5NF, labrasol, propylene carbonate, steareth 2, ST emulsifier 10, and ST elastomer-10.

In some embodiments, the pharmaceutically acceptable formulation is a cream comprising an oil-in-water base. In some embodiments, the pharmaceutically acceptable formulation is a cream comprising a water-in-oil base. In some embodiments, the cream comprises one or of white soft paraffin/petrolatum, mineral oil, propylene glycol, cyclomethicone, ST-cyclomethicone-5NF, emulsifier 10, ST-emulsifier, ST-elastomer-10, methylparaben, dibasic sodium phosphate, citric acid, propylparaben, and purified water. In some embodiments, the cream comprises white soft paraffin/petrolatum, mineral oil, propylene glycol, ST-cyclomethicone-5NF, ST-emulsifier, ST-elastomer-10, methylparaben, dibasic sodium phosphate, citric acid, propylparaben, and purified water. In some embodiments, the cream comprises white soft paraffin/petrolatum, mineral oil, propylene glycol, cyclomethicone, emulsifier 10, ST-elastomer-10, methylparaben, sodium phosphate dibasic anhydrous, citric acid anhydrous, propylparaben, and purified water.

Pharmaceutical compositions as disclosed herein may be administered to a subject. A therapeutically effective amount of a pharmaceutical composition as disclosed herein may be administered to a subject. The pharmaceutical composition may be administered through any mode of delivery as disclosed herein, e.g., eye drops or cream. The eye drops may be administered via an eyedropper. The pharmaceutical composition may be administered to an eye of a subject or a portion thereof. The pharmaceutical composition may be administered to an eyelid, e.g., an outer eyelid.

The subject may suffer from a Meibomian gland dysfunction. Meibomian gland dysfunction can lead to altered tear film composition, ocular surface disease, ocular and eyelid discomfort, and evaporative dry eye. Symptoms of Meibomian gland dysfunction comprise dryness, burning, itching, redness, crusty discharge, watery eyes, blurred vision, and light sensitivity.

In some embodiments, an administration of pharmaceutical compositions as disclosed herein to the subject may result in an increase in a size of the meibomian gland as compared to subjects who do not receive the pharmaceutical composition. In some embodiments, an administration of pharmaceutical compositions as disclosed herein to the subject may result in a decrease in meibomian gland atrophy as compared to subjects who do not receive the pharmaceutical composition. In some embodiments, an administration of pharmaceutical compositions as disclosed herein to the subject may result in regeneration of meibomian glands as compared to subjects who do not receive the pharmaceutical composition. In some embodiments, an administration of pharmaceutical compositions as disclosed herein to the subject may result in a reversal of age-associated meibomian gland atrophy as compared to subjects who do not receive the pharmaceutical composition. In some embodiments, an administration of pharmaceutical compositions as disclosed herein to the subject may result in an increase in a function of one or more meibocytes as compared to subjects who do not receive the pharmaceutical composition. For example, in some instances, the increase in function may comprises increase phosphorylation of Akt (or increased duration in activity for phosphorylation or Akt), IGF1R itself or other downstream signaling molecules that are phosphorylated when IGF1R is activated. In some embodiments, an administration of pharmaceutical compositions as disclosed herein to the subject may result in an increase in corneal proliferation and/or repair as compared to subjects who do not receive the pharmaceutical composition. In some embodiments, an administration of pharmaceutical compositions as disclosed herein to the subject may result in an increase in corneal healing as compared to subjects who do not receive the pharmaceutical composition. In some embodiments, an administration of pharmaceutical compositions as disclosed herein to the subject may result in an increase in IGF1 receptor (IGF1R) activation in the meibomian glands as compared to subjects who do not receive the pharmaceutical composition. In some embodiments, an administration of pharmaceutical compositions as disclosed herein to the subject may result in an increase duration of IGF1R activation in the meibomian glands as compared to subjects who do not receive the pharmaceutical composition. In some embodiments, an administration of pharmaceutical compositions as disclosed herein to the subject may result in an increase of lipid content of the meibomian glands as compared to subjects who do not receive the pharmaceutical composition.

The administration of the pharmaceutical composition to the subject suffering from Meibomian gland dysfunction may result in a median increase in surface area or volume of meibomian glands of the subject as compared to subjects who do not receive the pharmaceutical composition. In some embodiments, the increase in surface area or volume of meibomian glands within the inner eyelid surface of the subject is at least 5% compared to subjects who do not receive the pharmaceutical composition. In some embodiments, the increase in surface area or volume of meibomian glands of the subject is at least 6% compared to subjects who do not receive the pharmaceutical composition. In some embodiments, the increase in surface area or volume of meibomian glands of the subject is at least 7% compared to subjects who do not receive the pharmaceutical composition. In some embodiments, the increase in surface area or volume of meibomian glands of the subject is at least 8% compared to subjects who do not receive the pharmaceutical composition. In some embodiments, the increase in surface area or volume of meibomian glands of the subject is at least 9% compared to subjects who do not receive the pharmaceutical composition. In some embodiments, the increase in surface area or volume of meibomian of the subject is at least 10% compared to subjects who do not receive the pharmaceutical composition. In some embodiments, the increase in surface area or volume of meibomian glands of the subject is at least 15% compared to subjects who do not receive the pharmaceutical composition. In some embodiments, the increase in surface area or volume of meibomian glands of the subject is at least 20% compared to subjects who do not receive the pharmaceutical composition. In some embodiments, the increase in surface area or volume of meibomian glands of the subject is at least 30% compared to subjects who do not receive the pharmaceutical composition. In some embodiments, the increase in surface area or volume of meibomian glands of the subject is at least 40% compared to subjects who do not receive the pharmaceutical composition. In some embodiments, the increase in surface area or volume of meibomian glands of the subject is at least 50% compared to subjects who do not receive the pharmaceutical composition. In some embodiments, the increase in surface area or volume of meibomian glands of the subject is at least 60% compared to subjects who do not receive the pharmaceutical composition.

The administration of the pharmaceutical composition to the subject suffering from Meibomian gland dysfunction may result in a median increase in lipid content within Meibomian glands as compared to subjects who do not receive the pharmaceutical composition. In some embodiments, the increase in lipid content within Meibomian glands is at least 5% compared to subjects who do not receive the pharmaceutical composition. In some embodiments, the increase in lipid content within Meibomian glands is at least 6% compared to subjects who do not receive the pharmaceutical composition. In some embodiments, the increase in lipid content within Meibomian glands is at least 7% compared to subjects who do not receive the pharmaceutical composition. In some embodiments, the increase in lipid content within Meibomian glands is at least 8% compared to subjects who do not receive the pharmaceutical composition. In some embodiments, the increase in lipid content within Meibomian glands is at least 9% compared to subjects who do not receive the pharmaceutical composition. In some embodiments, the increase in lipid content within Meibomian glands is at least 10% compared to subjects who do not receive the pharmaceutical composition. In some embodiments, the increase in lipid content within Meibomian glands is at least 15% compared to subjects who do not receive the pharmaceutical composition. In some embodiments, the increase in lipid content within Meibomian glands is at least 20% compared to subjects who do not receive the pharmaceutical composition. In some embodiments, the increase in lipid content within Meibomian glands is at least 30% compared to subjects who do not receive the pharmaceutical composition. In some embodiments, the increase in lipid content within Meibomian glands is at least 40% compared to subjects who do not receive the pharmaceutical composition. In some embodiments, the increase in lipid content within Meibomian glands is at least 50% compared to subjects who do not receive the pharmaceutical composition. In some embodiments, the increase in lipid content within Meibomian glands is at least 60% compared to subjects who do not receive the pharmaceutical composition.

The administration of the pharmaceutical composition to the subject suffering from Meibomian gland dysfunction may result in a median increase in lipid quality within Meibomian glands as compared to subjects who do not receive the pharmaceutical composition. The lipid quality may be assessed by having a lower melting point (e.g., being less waxy) that lipids (e.g., meibum) from subjects who did not receive the pharmaceutical composition. Stated differently lipids (e.g., meibum) with a lower average melting point may have improved quality relative to lipids (e.g., meibum) with a higher average melting point. In some embodiments, the increase in lipid quality within Meibomian glands is at least 5% compared to subjects who do not receive the pharmaceutical composition. In some embodiments, the increase in lipid quality within Meibomian glands is at least 6% compared to subjects who do not receive the pharmaceutical composition. In some embodiments, the increase in lipid quality within Meibomian glands is at least 7% compared to subjects who do not receive the pharmaceutical composition. In some embodiments, the increase in lipid quality within Meibomian glands is at least 8% compared to subjects who do not receive the pharmaceutical composition. In some embodiments, the increase in lipid quality within Meibomian glands is at least 9% compared to subjects who do not receive the pharmaceutical composition. In some embodiments, the increase in lipid quality within Meibomian glands is at least 10% compared to subjects who do not receive the pharmaceutical composition. In some embodiments, the increase in lipid quality within Meibomian glands is at least 15% compared to subjects who do not receive the pharmaceutical composition. In some embodiments, the increase in lipid quality within Meibomian glands is at least 20% compared to subjects who do not receive the pharmaceutical composition. In some embodiments, the increase in lipid quality within Meibomian glands is at least 30% compared to subjects who do not receive the pharmaceutical composition. In some embodiments, the increase in lipid quality within Meibomian glands is at least 40% compared to subjects who do not receive the pharmaceutical composition. In some embodiments, the increase in lipid quality within Meibomian glands is at least 50% compared to subjects who do not receive the pharmaceutical composition. In some embodiments, the increase in lipid quality within Meibomian glands is at least 60% compared to subjects who do not receive the pharmaceutical composition.

The administration of the pharmaceutical composition to the subject suffering from Meibomian gland dysfunction may result in an increase in release of lipid from acini of the Meibomian glands as compared to subjects who do not receive the pharmaceutical composition. In some embodiments, the increase in release of lipid from acini of the Meibomian glands is at least 5% compared to subjects who do not receive the pharmaceutical composition. In some embodiments, the increase in release of lipid from acini of the Meibomian glands is at least 10% compared to subjects who do not receive the pharmaceutical composition. In some embodiments, the increase in release of lipid from acini of the Meibomian glands is at least 15% compared to subjects who do not receive the pharmaceutical composition. In some embodiments, the increase in release of lipid from acini of the Meibomian glands is at least 20% compared to subjects who do not receive the pharmaceutical composition. In some embodiments, the increase in release of lipid from acini of the Meibomian glands is at least 30% compared to subjects who do not receive the pharmaceutical composition. In some embodiments, the increase in release of lipid from acini of the Meibomian glands is at least 40% compared to subjects who do not receive the pharmaceutical composition. In some embodiments, the increase in release of lipid from acini of the Meibomian glands is at least 50% compared to subjects who do not receive the pharmaceutical composition. In some embodiments, the increase in release of lipid from acini of the Meibomian glands is at least 75% compared to subjects who do not receive the pharmaceutical composition. In some embodiments, the increase in release of lipid from acini of the Meibomian glands is at least 100% compared to subjects who do not receive the pharmaceutical composition. In some embodiments, the increase in release of lipid from acini of the Meibomian glands is at least 150% compared to subjects who do not receive the pharmaceutical composition. In some embodiments, the increase in release of lipid from acini of the Meibomian glands is at least 200% compared to subjects who do not receive the pharmaceutical composition. In some embodiments, the increase in release of lipid from acini of the Meibomian glands is at least 300% compared to subjects who do not receive the pharmaceutical composition.

The administration of the pharmaceutical composition to the subject suffering from Meibomian gland dysfunction may result in an increase in one or more pharmacodynamic effects, such as an increase in duration of phosphorylation of Akt in meibocytes as compared to subjects who do not receive the pharmaceutical composition. In some embodiments, the increase in duration of phosphorylation of Akt in meibocytes is at least 5% compared to subjects who do not receive the pharmaceutical composition. In some embodiments, the increase in duration of phosphorylation of Akt in meibocytes is at least 10% compared to subjects who do not receive the pharmaceutical composition. In some embodiments, the increase in duration of phosphorylation of Akt in meibocytes is at least 15% compared to subjects who do not receive the pharmaceutical composition. In some embodiments, the increase in duration of phosphorylation of Akt in meibocytes is at least 20% compared to subjects who do not receive the pharmaceutical composition. In some embodiments, the increase in duration of phosphorylation of Akt in meibocytes is at least 30% compared to subjects who do not receive the pharmaceutical composition. In some embodiments, the increase in duration of phosphorylation of Akt in meibocytes is at least 40% compared to subjects who do not receive the pharmaceutical composition. In some embodiments, the increase in duration of phosphorylation of Akt in meibocytes is at least 50% compared to subjects who do not receive the pharmaceutical composition. In another embodiment, the change in one or more pharmacodynamic effects may be increased phosphorylation of any downstream target resulting from activation of the IGF1R.

The pharmaceutical composition may comprise a polypeptide as disclosed herein. The polypeptide may comprise the sequence of any one of SEQ ID NOS: 1-8 or 12. The polypeptide may be an IGF-1 variant. The IGF-1 variant may comprise or consist of the sequence of any one of SEQ ID NOS: 2-8 or 12. The pharmaceutical composition may comprise an IGF-1 variant that has reduced affinity to at least one IGFBP relative to wild-type IGF-1 (SEQ ID NO: 1) to the IGFBP.

In some embodiments, the pharmaceutical composition does not comprise any additional phospholipidosis-inducing agent. In some embodiments, the pharmaceutical composition does not comprise any one of azithromycin or doxycycline.

### Method of Treatment, Administration, and Uses

Also provided herein are methods for treating a disease or a condition in a subject in need thereof. Also provided herein are methods for treating an eye disorder in a subject in need thereof. In some aspects, the method comprises administering a pharmaceutical composition provided herein. In some aspects, the method comprises administering a polypeptide provided herein. In some aspects, the method comprises administering an IGF-1 variant provided herein. In some embodiments of the methods disclosed herein, the pharmaceutical composition is administered locally. In some embodiments, the subject is human. In some embodiments, a therapeutically effective amount of the pharmaceutical composition provided herein is administered.

Also provided herein is use of the pharmaceutical composition provided herein in treating an eye disorder.

The subject can have a certain disease or a condition in need of treatment provided by the present disclosure. The disease or condition can include dry eye disease, meibomian gland dysfunction, and/or Sjorgren's syndrome.

For treatment, the amount of the pharmaceutical composition provided herein administered is an amount effective in producing the desired effect, for example, treatment or amelioration of the effects and/or symptoms of an eye disorder in a subject in need thereof. An effective amount can be provided in one or a series of administrations of the pharmaceutical composition provided herein.

Subjects suffering from the eye disorder can be identified by any or a combination of diagnostic or prognostic assays known in the art.

Methods for treating a subject in need thereof can further comprise sequentially, separately, or simultaneously administering to the subject at least one additional therapy, e.g., artificial tears or punctal plugs.

In any case, the multiple therapeutic agents can be administered in any order or even simultaneously. If simultaneously, the multiple therapeutic agents can be provided in a single, unified form, or in multiple forms (by way of example only, either as a single pill or as two separate pills). One of the therapeutic agents can be given in multiple doses, or both can be given as multiple doses. If not simultaneous, the timing between the multiple doses can vary from more than zero weeks to less than four weeks. In addition, the combination methods, compositions and formulations are not to be limited to the use of only two agents.

### Kits

The present disclosure provides kits comprising a pharmaceutical composition as disclosed herein, and an eyedropper. The eyedropper may be configured for a delivery of the pharmaceutical composition as an eyedrop.

### EXAMPLES

### Example 1: In Vitro Potency of Wild-Type IGF-1 and IGF-1 Variants in the Presence and Absence of IGFBPs

### Materials and Methods

### In Vitro Potency Assay in DU145 Cells

15×10⁶ DU145 cells (ATCC #HTB-81) frozen down at passage 5 (P5) were reseeded in a T175 culture flask containing DMEM/F12 (1:1) medium (Gibco 11320-033) supplemented with 10% FBS (Gibco 10437-028) and 10 ug/mL Gentamicin (Gibco 15710-064) (“culture medium”) and expanded. At 80-90% confluency, cells were trypsinized, manually counted using a hemocytometer, resuspended at 3×10⁵ cells per mL in culture medium, and then reseeded in 24-well plates (Corning 3524) in 0.5 mL aliquots per well (150,000 cells/well). 14-16 hours after reseeding, the culture medium was removed, and following a wash with 1 mL PBS, cultured for 6 h in 225 μL serum-free culture medium supplemented with 0.2% BSA (Millipore #A3059). 25 μL aliquots of the following growth factor dilution series were added to the cells in duplicate for a final concentration in the well of: 457, 137, 41, 12.5, 3.7, 1.11, and 0.33 nM IGF-1 (SEQ ID NO: 1), IGF-1 Ea (SEQ ID NO: 7), IGF-1 Des1-3 R37X (SEQ ID NO: 6), IGF-1 Des1-3 (SEQ ID NO: 8), and IGF-1 E3R (SEQ ID NO: 4). After 15 min incubation at 37° C., medium was aspirated and cells lysed in 150 μL 1×PTR buffer (Extraction Buffer 5× PTR, Abcam ab193970) containing 1× Extraction Enhancer Buffer (Extraction Enhancer Buffer 50×, Abcam ab193971) and protease/phosphatase inhibitors (one Pierce mini tablet [#A32959] per 10 mL 1×PTR). After 15-30 min incubation on ice, plates were vortexed, and lysates transferred to Eppendorf tubes and stored at −80° C. for assessment of IGF1R-AKT signaling strength using the Human/Mouse/Rat Phospho-Akt (S473) Pan Specific DuoSet IC ELISA (RnD #DYC887B).

Culture of IGFBP and IGF-1 in DU145 Cells

To measure the in vitro potency of IGF-1 and IGF-1 variants in the presence of IGFBP, DU145 cells were prepared as described above. During this time, IGFBPs in a 1:1, 2:1 or 4:1 molar ratio were incubated with 250 nM rhIGF1 (SEQ ID NO: 1), rhIGF1 LR3 (SEQ ID NO: 12), or rhIGF1 E3R (SEQ ID NO: 4). After 1 hour incubation at room temperature, complexes containing a final concentration of 25 nM of IGF1 variants were added to the cells in triplicate.

### Results

The dose-dependent effects of IGF-1 (SEQ ID NO: 1), IGF-1 Ea (SEQ ID NO: 7), IGF-1 Des1-3 R37X (SEQ ID NO: 6), IGF-1 Des1-3 (SEQ ID NO: 8), and IGF-1 E3R (SEQ ID NO: 4) on AKT S473 phosphorylation were measured in DU145 cells. The EC₅₀ value of IGF-1 was 6.0 nM. The EC₅₀ value of IGF-1 Ea was 8.7 nM. The EC₅₀ value of IGF-1 Des 1-3 R37X was 4.1 nM. The EC₅₀ value of IGF-1 Des 1-3 was 2.3 nM. The EC₅₀ value of IGF-1 E3R was 4.5 nM, as shown in FIG. 1. It suggests that wild-type IGF-1 and the tested IGF-1 variants have similar EC₅₀ values in vitro.

A literature search was conducted to determine differences in the ability of various IGF1 mutants to bind IGFBPs. Results of this search are shown in FIG. 2. In L6 rat myoblast conditioned media containing multiple binding proteins, competition assays report reduction in BP affinity for IGF-1 LR3 (SEQ ID NO: 12), LG3 (long IGF-1 E3G), long IGF-1, and IGF-1 Des 1-3 (SEQ ID NO: 3) to be 690 times, 112 times, 5.5 times and 38 times respectively (see Francis, G L et al., 8(3) J. Mol. Endocrinol. 213-223, 1992). In a separate study using bovine IGFBP2, IGF-1 E3R (SEQ ID NO: 4) and IGF E3G were reported to have reduction in binding affinity for IGFBP2 by 230 times and 59 times, respectively (see King, R. et al., 8 J. Mol. Endocrinol. 29-41, 1992). It suggests that some IGF-1 variants such as IGF-1 Des 1-3 (SEQ ID NO: 3), IGF-1 E3R (SEQ ID NO: 4), and IGF-1 LR3 (SEQ ID NO: 12) have reduced affinity for IGFBPs.

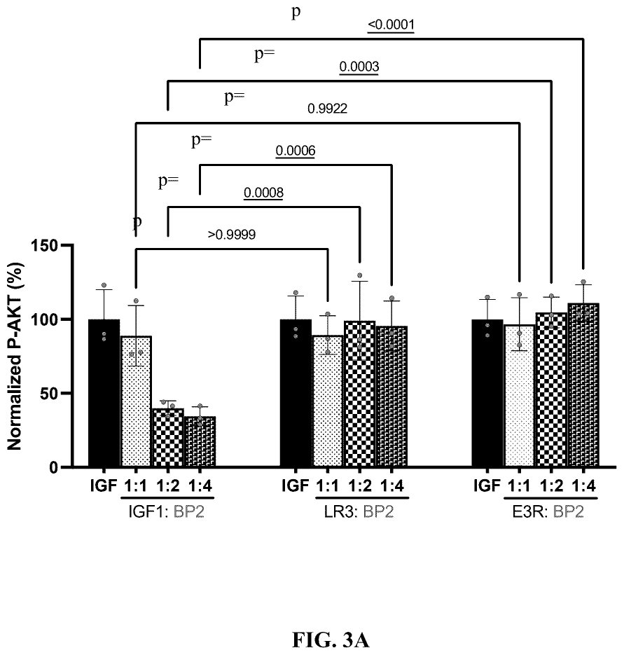

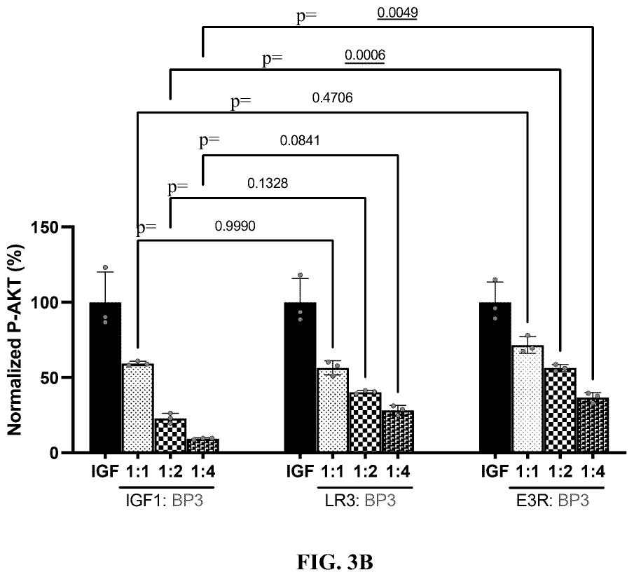

To determine the potency of IGF-1 variants in the presence of IGFBPs, pAKT levels measured in DU145 cells after preincubation of IGFBP2 or IGFBP3 with wild-type IGF-1 (SEQ ID NO: 1), IGF-1 LR3 (SEQ ID NO: 12), or IGF-1 E3R (SEQ ID NO: 4) at an IGF-1 proteins to IGFBPs ratio of 1:1, 1:2, or 1:4. FIGS. 3A and 3B suggest that IGF-1 LR3 (SEQ ID NO: 12) and IGF-1 E3R (SEQ ID NO: 4) are less inhibited by IGFBP2 and IGFBP3 compared to wild-type IGF-1 (SEQ ID NO: 1).

### Example 2: Wild-Type IGF-1 Enhances Growth of Spheroids

### Materials and Methods

In Vitro Potency Assay (pAKT Assay)

5×10⁶ immortalized human meibomian gland epithelial cells (IHMGECs: ATCC #CRL-3472) frozen down at passage 5 (P5) were reseeded in a T75 culture flask containing KSFM medium (Gibco: #10724-011) supplemented with 5 μg/l human recombinant EGF (Gibco #10450-013) and bovine pituitary extract (BPE) (Gibco #13028-014), MEM NEAA (Gibco #11140-050) Pen Strep (Gibco #15140-122) (“proliferation medium”) and expanded. At 90-95% confluency, cells were trypsinized, counted using an EVE automated cell counter from NanoEntek, resuspended at 4×10⁵ cells ml in culture medium, and then reseeded in 12-well plates (ThermoFisher #FB012928) in 1-ml aliquots per well. 36-42 hours after reseeding, the culture medium was removed, and following two washes with 1 ml PBS, cultured for 6 h in 450 μl DMEM/F12 (1:1) medium (Gibco: #11320-033) supplemented with Pen Strep (Gibco #15140-122) and 0.2% BSA (Millipore #A3059) (“serum starvation medium”). 50 μl aliquots of the following rh-IGF-1 (SEQ ID NO: 1) dilution series were added to the cells in duplicate: 10000, 1000, 100, 10, 1, 0.1, 0 nM. After 15 min incubation at 37° C., culture medium was aspirated, and cells lysed in 100 μl M-PER (Thermo Scientific #78501) containing protease/phosphatase inhibitors (one Pierce mini tablet [#A32961] per 10 ml M-PER). After 15-30 min incubation on ice, plates were vortexed, and lysates transferred to Eppendorf tubes and stored at −80° C. for assessment of IGFR1-AKT signaling strength using the phospho-Akt (S473) Pan Specific DuoSet IC ELISA (RnD #DYC887B-2). ELISAs were performed in 384-well plates to the manufacturers' recommendation with minor modifications. Briefly, wells of 384-well plates were coated with 25 μl 6 μg/ml phospho-Akt1 (S473) capture antibody (#841692) in PBS (25 μl per 384-well), sealed and placed overnight at RT. Wells were washed 4× with 100 μl PBS containing 0.05% Tween 20, and blocked with 50 μl PBS containing 2% BSA for 1-2 h at room temperature (RT). Wells were washed 4× with 100 μl PBS containing 0.05% Tween 20 and incubated with 25 μl of lysate or P-AKT standard for 2 h at RT. Wells were washed 4× with 100 μl PBS containing 0.05% Tween 20 and then incubated with 25 μl 100 ng/ml phospho-Akt1 (S473) detection antibody (#843081) diluted in PBS containing 1% BSA for 1 h at RT. Wells were washed 4× with 100 μl PBS containing 0.05% Tween 20 and incubated with 25 μl Streptavidin-HRP A (RnD #890803) diluted 1:200 in PBS containing 1% BSA for 20 min at RT. Wells were washed 4× with 100 μl PBS containing 0.05% Tween 20 and incubated with 25 μl TMB substrate solution (Abcam TMB ELISA Substrate High Sensitivity (ab171523; lot GR3427893-1)), and incubated for 10-20 min at RT. 12.5 μl stop solution (RnD #895926 from Ancillary Kit 2) was added and the OD450 of each well measured using a BMG Labtech CLARIOstar® Plus Microplate Reader. EC₅₀ values were calculated using Prism 9.

### In Vitro Human Meibomian Gland Epithelial Cell Growth Assays

Live-Cell imaging assays. 5×10⁶ immortalized human meibomian gland epithelial cells (IHMGECs: ATCC #CRL-3472) frozen down at passage 5 (P5) were reseed in a T75 culture flask containing proliferation medium and expanded. At 90-95% confluency, cells were trypsinized, counted using an EVE automated cell counter from NanoEntek, resuspended at 1×10⁵ cells ml in proliferation medium, and then reseeded in two 12-well plates (ThermoFisher #FB012928) in 1-ml aliquots per well. 36 hours after reseeding, the proliferation medium was removed, and following two washes with 1 ml PBS, cultured in 500 μl serum starvation medium supplemented with 0, 0.1, 1, 10, 100 or 1000 nM human LR3-IGF-1 (PeproTech: #100-11R3) (4 independent wells for each condition). At 24-hour intervals, cells in the center of each well were imaged using an Eclipse Ti microscope (Nikon) with a 10× Plan Fluor Ph1 lens (Nikon) and an iXon Life 888 EMCCD camera.

CellTiter-Glo Luminescent Cell Viability Assay. 5×10⁶ immortalized human meibomian gland epithelial cells (IHMGECs: ATCC #CRL-3472) frozen down at passage 5 (P5) were reseeded in a T75 culture flask containing proliferation medium and expanded. At 90-95% confluency, cells were trypsinized, counted using an EVE automated cell counter from NanoEntek, resuspended at 1×10⁵ cells ml in proliferation medium, and then reseeded in two 12-well plates (ThermoFisher FB012928) in 1-ml aliquots per well. 36 hours after reseeding, the proliferation medium was removed, and following two washes with 1 ml PBS, cultured in 500 μl serum starvation medium supplemented with 0, 0.1, 1, 10, 100 or 1000 nM human IGF-1 (R&D 291-G1). 58 hours later, the medium on each well was replaced with 200 μl DMEM/F12 and an equal volume of CellTiter-Glo® Reagent. After 10 minutes on an orbital shaker two 100 μl aliquots of each 12 well were transferred to a 96-well flat-bottom black fluotrac plate (Greiner #655076) and luminescence measured at 560-580 nm using a BMG Labtech CLARIOstar® Plus Microplate Reader. As a measure of cell IGF1-mediated proliferation, RLU values of IGF1-treated IHMGEC were normalized to IHMGEC cultured in the absence of human IGF1.

### Results

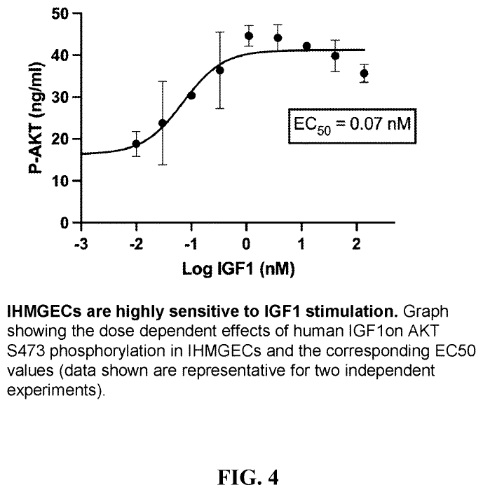

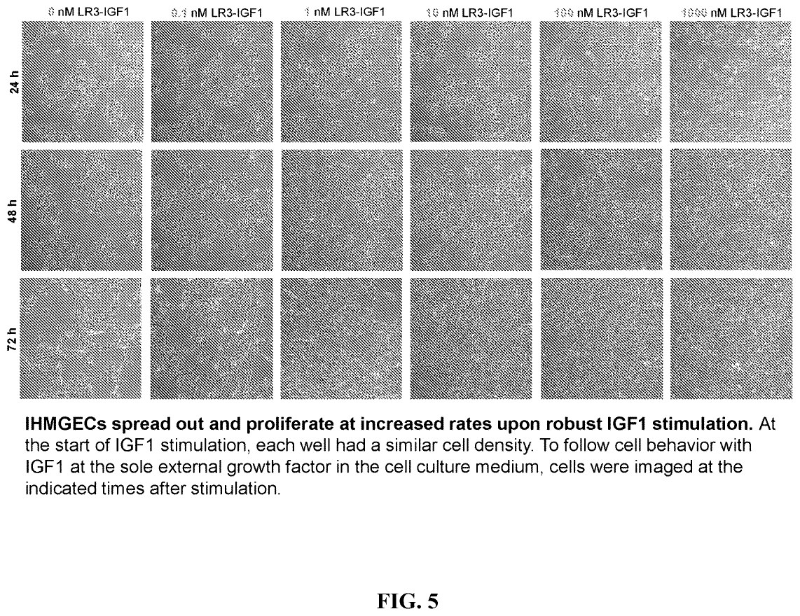

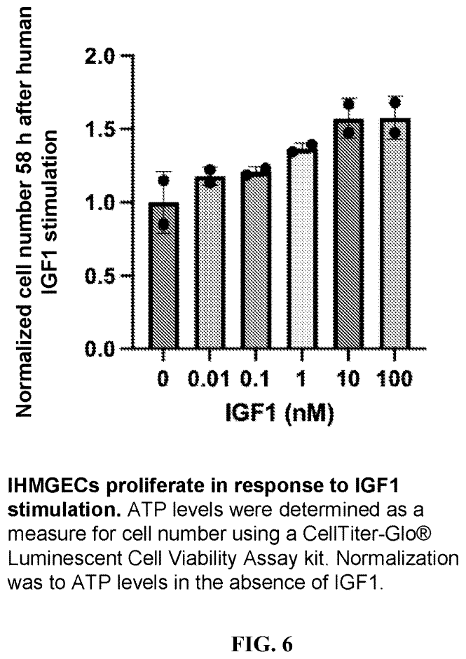

FIG. 4 depicts the dose-dependent effects of IGF-1 on AKT S473 phosphorylation in IHMGECs. The EC₅₀ value for IGF-1 is about 0.07 nanomolar (nM). FIG. 5 depicts the results of live cell imaging assays: IHMGECs spread out and proliferate at increased rates upon IGF-1 stimulation in a dose-dependent manner. FIG. 6 depicts the results of human IGF-1 stimulation in IGHMECs following the cell viability assay: the cells proliferate in response to IGF-1 in a dose-dependent matter.

### Example 3: IGF1 E3R Enhances Growth of Spheroids More Strongly than Wild-Type IGF-1

### Materials and Methods

### 3D Culture of IHGMGE Cells

2500 immortalized human meibomian gland epithelial cells (IHMGEC, ATCC, #CRL-3472) were seeded per well of a 24-well plate in a drop of 50 μL matrix (Matrigel, Corning #354230) covered by proliferation medium (Keratinocyte Serum Free Medium (Gibco, #17005042) supplemented with 50 μg/mL bovine pituitary extract (Gibco, #13028-014), 5 ng/ml recombinant human epidermal growth factor (EGF, Gibco, #10450-013), 10 μg/mL Gentamicin (Gibco, #15710-064) and 1% Pen/Strep (Gibco, #15140-122)). Spheroids were grown in proliferation medium for eight days, followed by a differentiation stage of seven days in presence of differentiation medium (DMEM: F12 medium (Gibco #11320-033) supplemented with 10 μg/mL Gentamicin (Gibco, #15710-064), 1% Pen/Strep (Gibco, #15140-122), 20 μM Rosiglitazone (Sigma Aldrich, #R2408), plus or minus various amounts of recombinant human insulin-like growth factor-1 (rhIGF1, PeproTech #100-11) or IGF1-E3R (SEQ ID NO: 12). N=2 per condition.

On day six of differentiation, spheroids were fixed in 4% PFA and processed for immunofluorescence using an antibody against Krt5 (Purified anti-Keratin 5 Polyclonal Chicken Antibody, Biolegend #905903) and detected with a AF594-labeled secondary anti-chicken antibody (Goat anti-Chicken IgY (H+L) Secondary Antibody, Alexa Fluo 594, Thermo #A-11042). For images of 2D and 3D spheroid cultures, brightfield images were collected with transmitted light.

### Measurement of Spheroids' Growth of IHGMGE Cells

IHMGE cells were cultured as described above. As a measure of proliferation, Fiji 2 by ImageJ was used to measure the diameter of each spheroid at day 7 of differentiation. To this end, a z-stack of 25 bright field images spanning a total of 1 mm were taken and maximum image projection was used to generate one final image that was used for measurement.

### Measurement of Lipid Production in the Spheroids

IHMGE cells were cultured as described above. On day six of differentiation, 1:3000 LipidTOX Green neutral lipid stain (Invitrogen, #H34475) was added to the wells. As a measure of lipid production, at day seven of differentiation a z-stack of 25 4× images spanning 1 mm were taken with a fluorescent microscope with filter sets appropriate for Alexa Fluor 488 dye or fluorescein. After maximum image projection, the mean signal per spheroid was calculated using QuPath software (https://qupath.github.io/).

### Quantification of IGFBPs in the Spheroids

IHMGE cells were cultured as described above. On day six of differentiation, replicates were pooled and lysed in 200 μl of 2× Extraction Buffer (5×PTR, Abcam #, ab193970) and vortexed extensively. Lysates were then frozen at −80 degrees. For analysis, protein quantitation was performed on each sample using Pierce Detergent Compatible Bradford Assay Kit (Thermo, #23246) and analyzed by Human IGF Signaling Array C1 Ray Biotech, #AAH-IGF-1-4) according to the manufacturer's instructions.

### Results

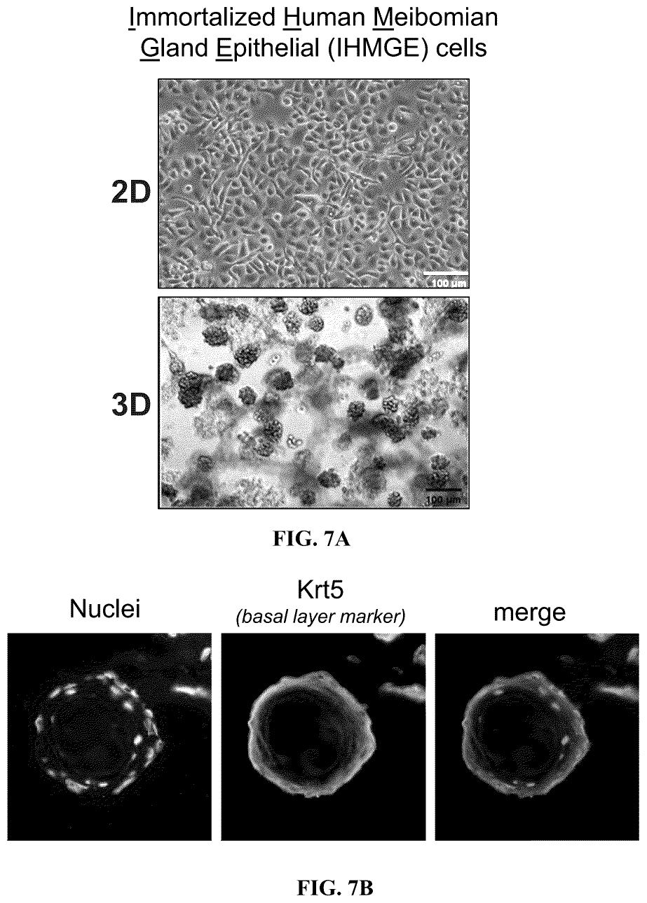

IHMGE cells grown in 3D culture under conditions promoted differentiation develop into spheroids structures resembling Meibomian gland Acini, as shown in FIGS. 7A and 7B. Spheroids expressed markers of the meibomian gland acini basal compartment including Krt5 and exhibit proliferation in the basal compartment, as shown in FIGS. 7A and 7B.

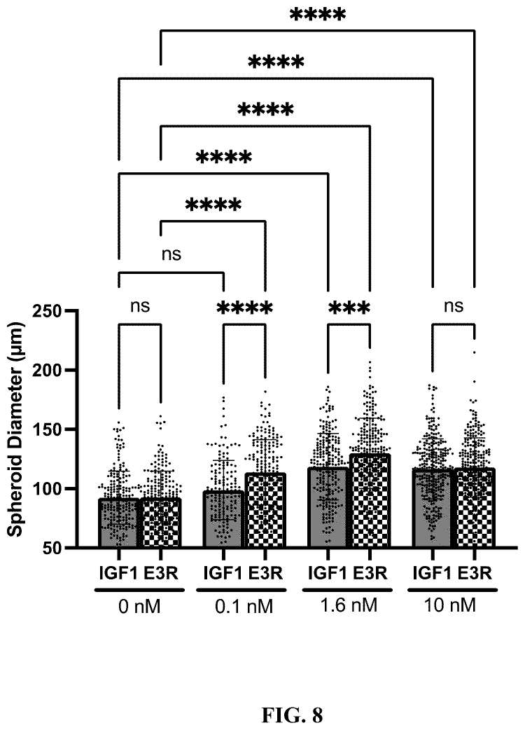

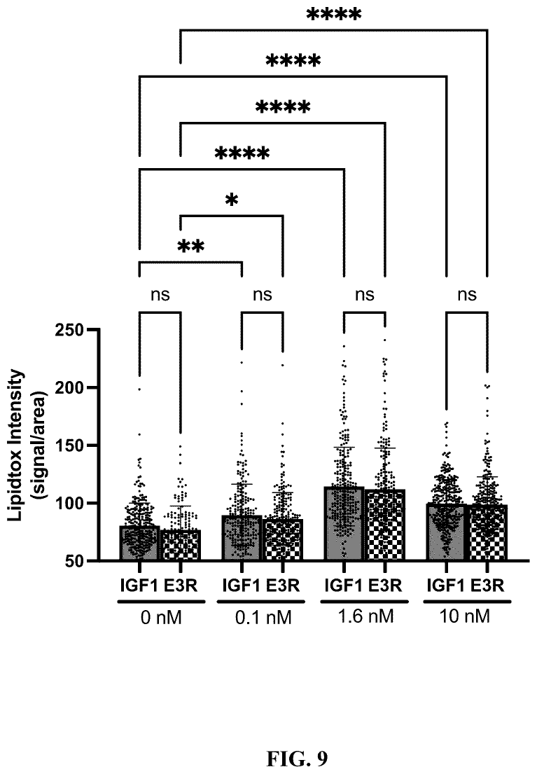

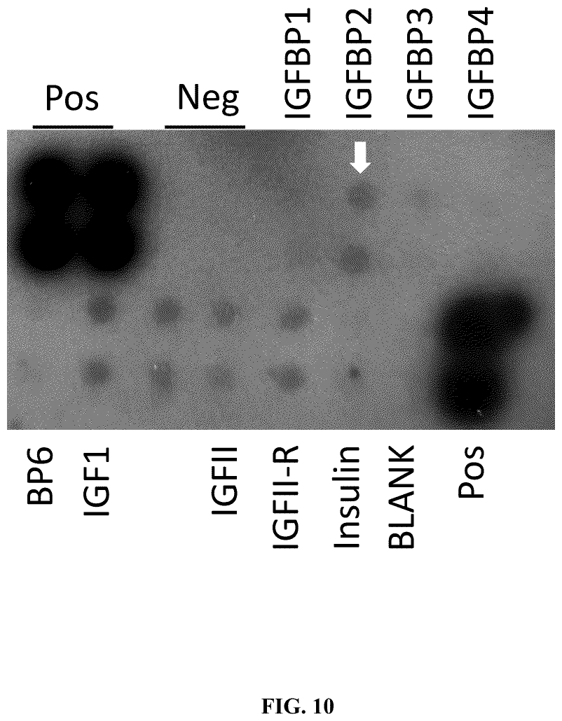

The perimeters of spheroids cultured in the presence of 0 nM, 0.1 nM, 1.6 nM, or 10 nM of IGF-1 or IGF-1 E3R were measured on day six of differentiation. FIG. 8 provides that spheroids grown in the presence of IGF-1 E3R were significantly larger than those grown with wild-type IGF-1. Furthermore, lipidtox intensity of spheroids cultured in the presence of 0 nM, 0.1 nM, 1.6 nM, or 10 nM of IGF-1 or IGF-1 E3R were measured on day seven of differentiation. IGF-1 and IGF-1 E3R proportionally increase Lipid content in spheroids, as shown in FIG. 9. Cells were lysed for quantification of IGFBPs. FIG. 10 provides that IGFBP2 was expressed higher compared to IGFBP1, IGFBP3, and IGFBP4 in IHMGE spheroid cultures.

### Example 4: IGF1 Treatment Upregulates Genes Involved in Fatty Acid Transport, Lipid Synthesis, and Meibogenesis

### Materials and Methods

2500 immortalized human meibomian gland epithelial cells (IHMGEC, ATCC, #CRL-3472) were seeded per well of a 24-well plate in a drop of 50 μL matrix (Geltrex, ThermoFisher #A1413201) covered by proliferation medium (Keratinocyte Serum Free Medium (Gibco, #17005042) supplemented with 50 μg/mL bovine pituitary extract (Gibco, #13028-014), 5 ng/ml recombinant human epidermal growth factor (EGF, Gibco, #10450-013), 10 μg/mL Gentamicin (Gibco, #15710-064) and 1% Pen/Strep (Gibco, #15140-122)). Spheroids were grown in proliferation medium for 14 days, followed by a differentiation stage of four days in presence of differentiation medium (DMEM: F12 medium (Gibco #11320-033) supplemented with 10 μg/mL Gentamicin (Gibco, #15710-064), 1% Pen/Strep (Gibco, #15140-122), 20 μM Rosiglitazone (Sigma Aldrich, #R2408), plus or minus 100 nM recombinant human insulin-like growth factor-1 (rhIGF1, PeproTech #100-11).

RNA extraction from 4-day differentiated spheroid cultures was performed by TRIzol extraction (Invitrogen #15596026) followed by an RNA clean-up step with RNA Clean & Concentrator-5 (Zymo Research #R1013) according to manufacturers' instructions. In brief, n=4 samples per condition. The droplets of matrix containing the spheroids were transferred to an Eppendorf tube and 500 μL TRIzol was added to lyse the cells, then 100 μL chloroform was added and the aqueous phase was transferred to an RNase-free tube and mixed with equal volume of 100% ethanol. The mixture was cleaned up with the Zymo-Spin IC Column, including a DNase I treatment, all according to protocol. RNA quality and quantity were assessed with a nanodrop (A260/A280=1.8-2, A260/A230>=1.8) prior to submitting total RNA samples to Novogene. At Novogene, RNA purity and integrity was confirmed. Then, a mRNA library with poly A enrichment was prepared and subsequently tested for its quality (Library QC). Next, a 150 bp paired-end sequencing strategy was used to sequence the library with the Illumina NovaSeq PE150 platform (6 G of raw data per sample), and the quality of the resulting data was also checked (Data QC).

Fastq files of paired-end RNA-Seq reads were aligned with STAR v. 2.6.0a (Dobin, A. et al., 29(1) Bioinformatics 15-21, 2013) against the reference genome hg38 (Schneider, V. A. et al., 27(5) Genome research 849-864, 2017). Gene level counts from read pairs were obtained using FeatureCounts v. 2.0.6 (Liao, Y. et al., 30(7) Bioinformatics 923-930, 2014) from the SubRead package. Differential expression analysis was performed using R package DESeq2 v.1.40.2 (Love, M. I. et al., 15(12) Genome biology 550, 2014). after removing genes with average raw counts <10. Gene Set Enrichment Analysis (GSEA) (Subramanian, A. et al., 102(43) Proc Natl Acad Sci USA. 15545-15550, 2005). was performed to detect enriched pathways against human gene sets from MSigDB (Liberzon A. et al., 27(12) Bioinformatics 1739-1740, 2011). using gene lists ranked in descending order by log2FoldChange. Overrepresentation analysis was performed using the Fisher's exact test method for selected up-(log2FoldChange >1, adjusted p-value <0.25) and down-regulated (log2FoldChange <−1, adjusted p-value <0.25) genes against gene sets related to meibogenesis gathered from literature. Morpheus (Broad Institute) was used to generate heatmaps.

### Results

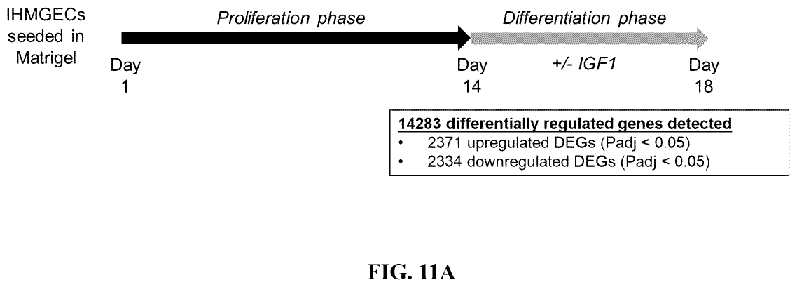

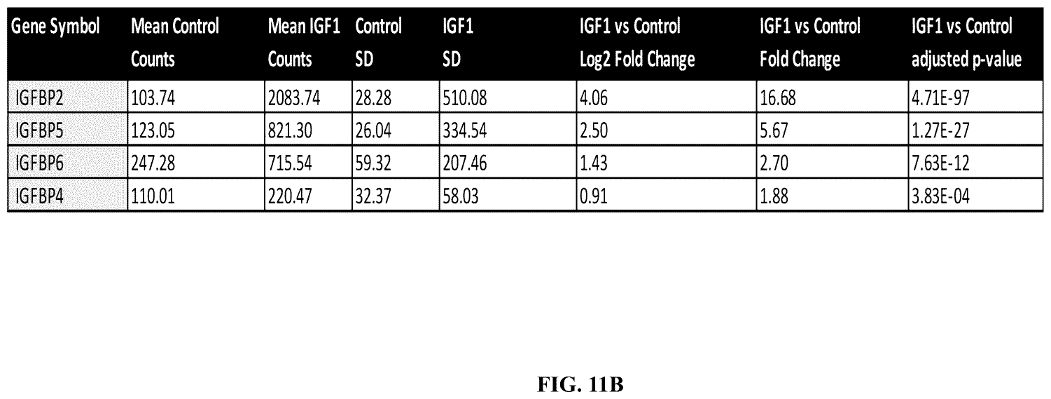

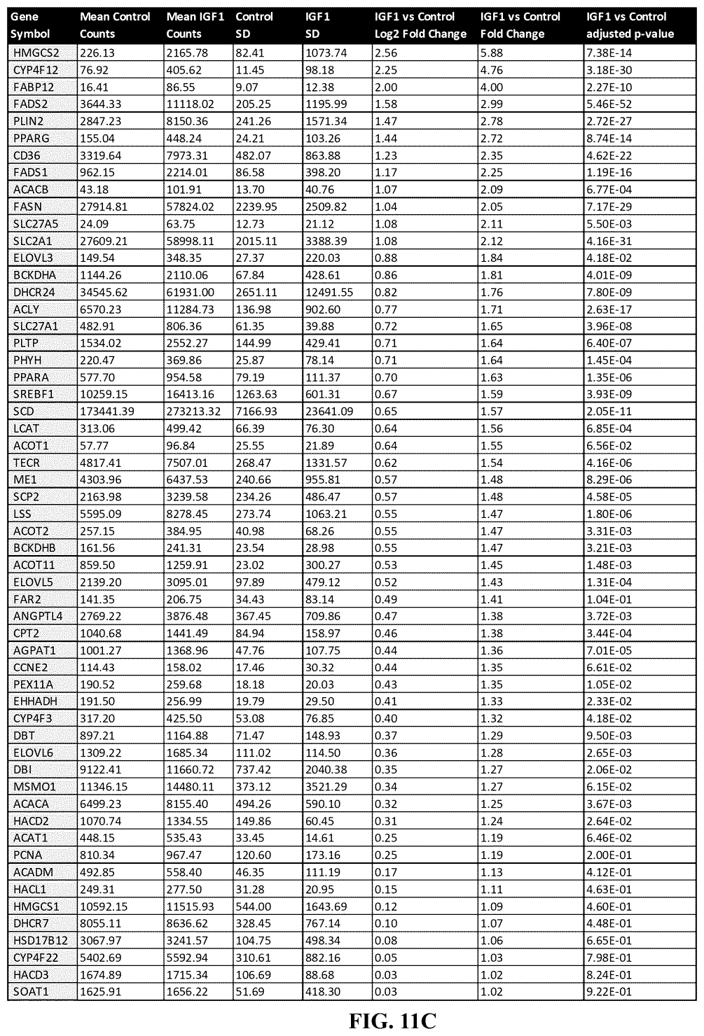

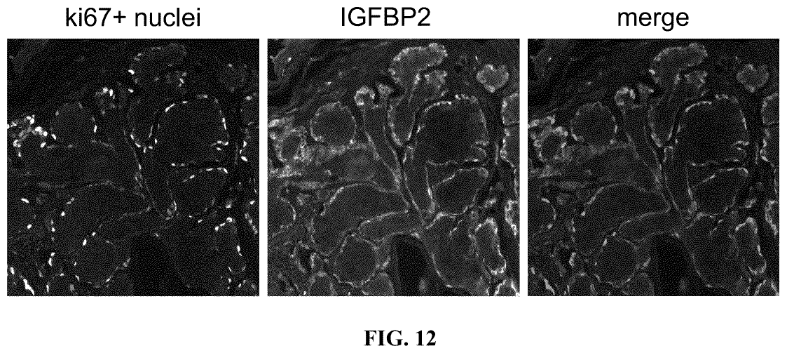

To perform transcriptional analysis of the effects of wild-type IGF1 treatment on IHMGE spheroids, IHMGE cells were seeded in Matrigel and differentiated for 4 days in the presence of rosiglitazone or rosiglitazone plus IGF1. RNA sequencing technology was used to determine the effects of wild-type IGF1 on gene expression in IHMGE spheroid. FIG. 11A illustrates the flowchart of the study design. Treatment of IHMGE spheroids with wild-type IGF1 significantly upregulates four IGFBPs, which are IGFBP2, IGFBP5, IGFBP4, and IGFBP6, as shown in FIG. 11B. Among them, IGFBP2 was the most significantly upregulated IGFBP in response to treatment with wild-type IGF1, as shown in FIG. 11B. Treatment of IHMGE spheroids with wtIGF1 significantly upregulates genes involved in fatty acid transport, lipid synthesis, and meibogenesis, as shown in FIG. 11C. Gene sets of interest were adapted from Butovich I., 163 Exp Eye Res. 2-16, 2017.

### Example 5: In Vivo Effects of IGF-1 Variants on Mouse Meibomian Glands

### Materials and Methods

### Animal Studies

Wild-type (WT) female C57BL/6J (Jackson Labs, Strain #000664) aged 2 months or 1-1.5 years of age were purchased. All animal studies were conducted in compliance with the relevant protocols.

### Dosing

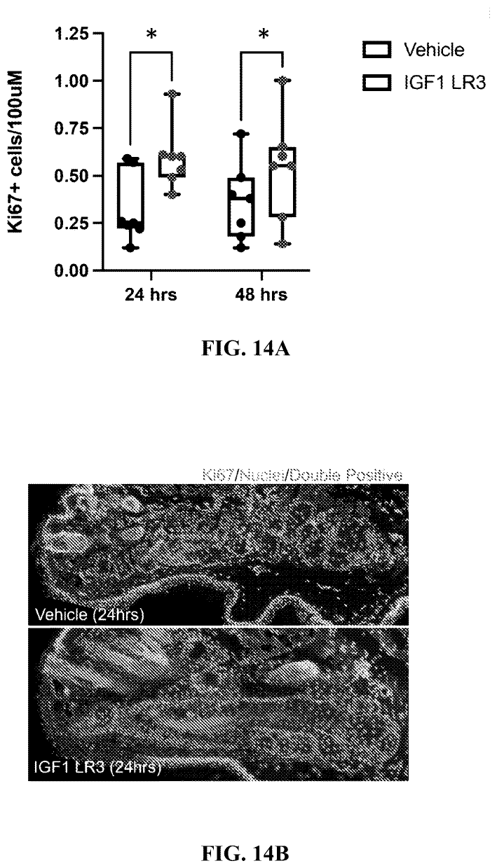

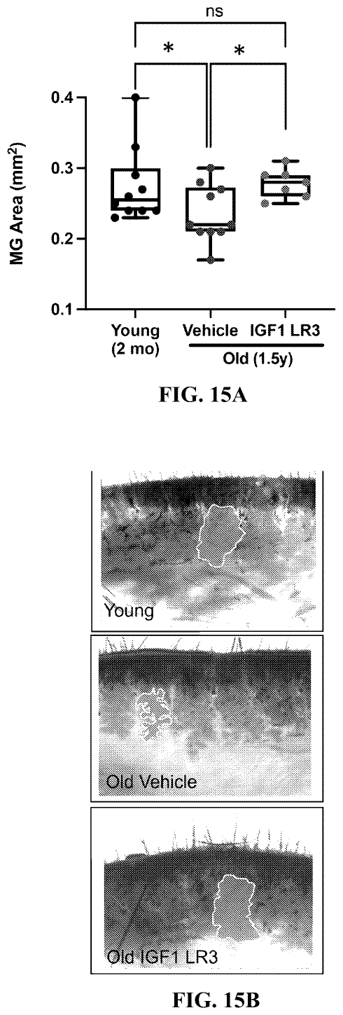

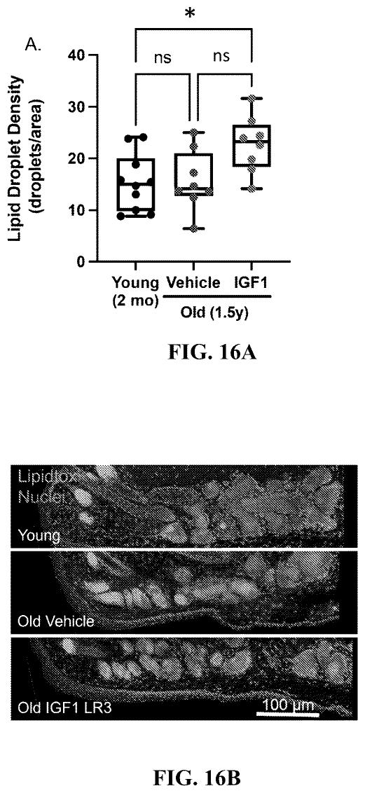

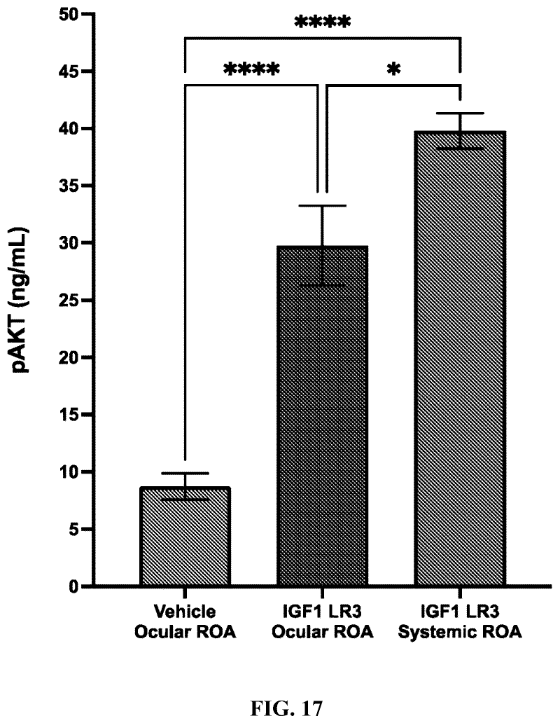

For dosing studies, mice were administered vehicle (Endotoxin-free PBS, EMD Millipore, TMS-012-A) or 10 milligram/kilogram (mg/kg) IGF-1 LR3 (SEQ ID NO: 12) resuspended in vehicle. Animals were dosed using insulin syringes (BD, 329424). Mice were given 2 intraperitoneal (IP) doses of IGF-1 LR3, 12 hours apart and sacrificed as described below at either 24 or 48 hours post dosing (results in FIGS. 14A and 14B). Mice were dosed daily IP with vehicle or 10 mg/kg IGF-1 LR3 for 5 days in a row followed by a 2 day holiday for a total of 4 weeks, then sacrificed as described below (results in FIGS. 15A, 15B, 16A, and 16B). Animals were dosed with a single IP dose of 10 mg/kg IGF-1 LR3 or given a 10 μl eyedrop of vehicle of IGF-1 LR3 at a concentration of 5 mg/ml, then sacrificed as described below (results in FIG. 17).

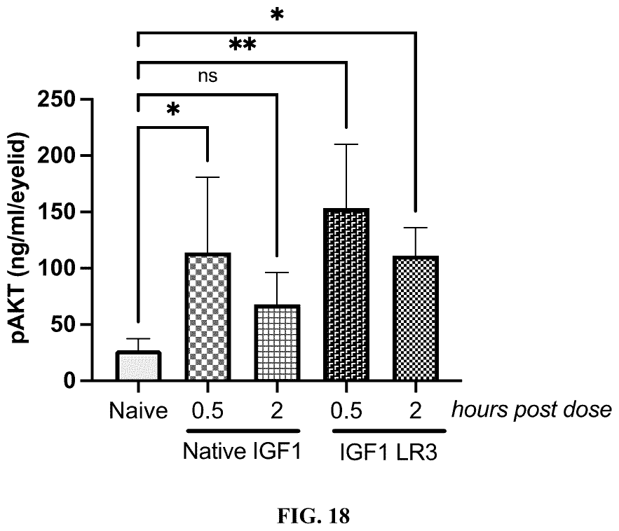

In FIG. 18, animals were administered a 10 μl eyedrop containing equimolar concentration based on molecular weight of either wild-type IGF-1 (SEQ ID NO: 1) at 1 mg/ml or IGF-1 LR3 (SEQ ID NO: 12) at 1.27 mg/ml then sacrificed as described previously at 0.5 hours or 2 hours post dosing. Eyelids were removed and frozen on dry ice then stored at −80° C. until analysis in P-AKT ELISA.

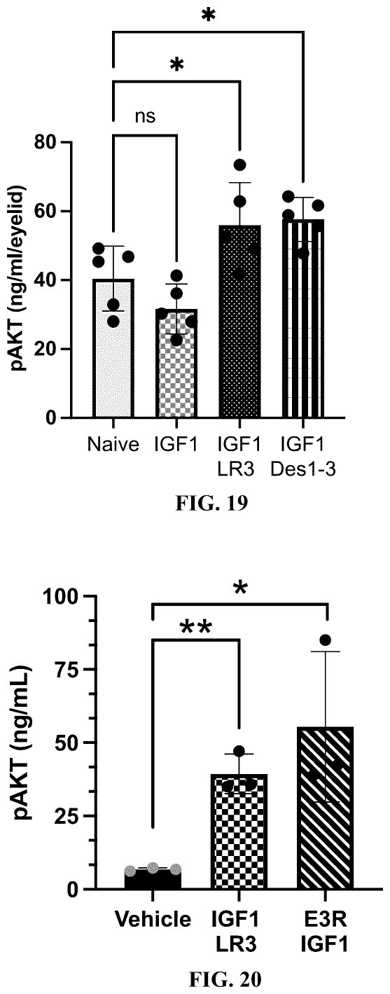

In FIG. 19, animals were administered a 10 μl eyedrop containing equimolar concentration based on molecular weight of either wild-type IGF-1 (SEQ ID NO: 1) at 1 mg/ml, IGF-1 LR3 (SEQ ID NO: 12) at 1.27 mg/ml, or rhIGF-1 Des1-3 (SEQ ID NO: 3) at 1 mg/ml then sacrificed as described previously at 2 hours post dosing. Eyelids were removed and frozen on dry ice then stored at −80° C. until analysis in P-AKT ELISA.

In FIG. 20, animals were administered a 10 μl eyedrop containing equimolar concentration based on molecular weight of either IGF-1 LR3 (SEQ ID NO: 12) at 1.27 mg/ml IGF-1 E3R (SEQ ID NO: 4) at 1 mg/ml then sacrificed as described previously at 1 hour post dosing. Eyelids were removed and frozen on dry ice then stored at −80° C. until analysis in P-AKT ELISA.

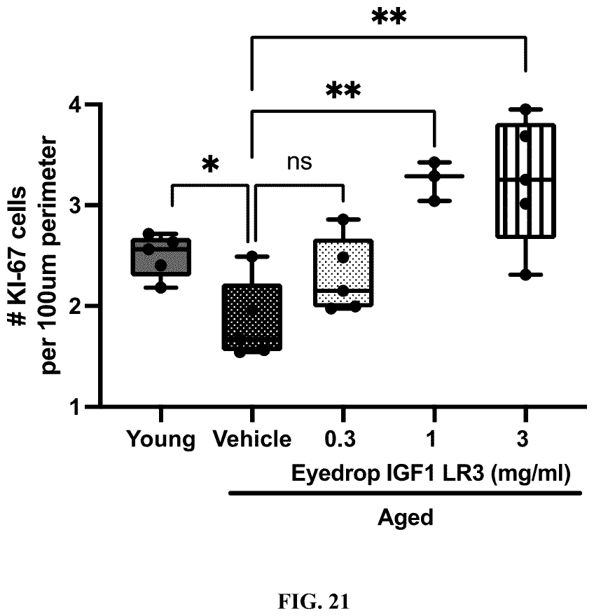

In FIG. 21, animals were administered a 10 μl eyedrop containing IGF-1 LR3 (SEQ ID NO: 12) at 0.3, 1 or 3 mg/ml every day for 2 weeks and sacrificed 24 hours after the last dose.

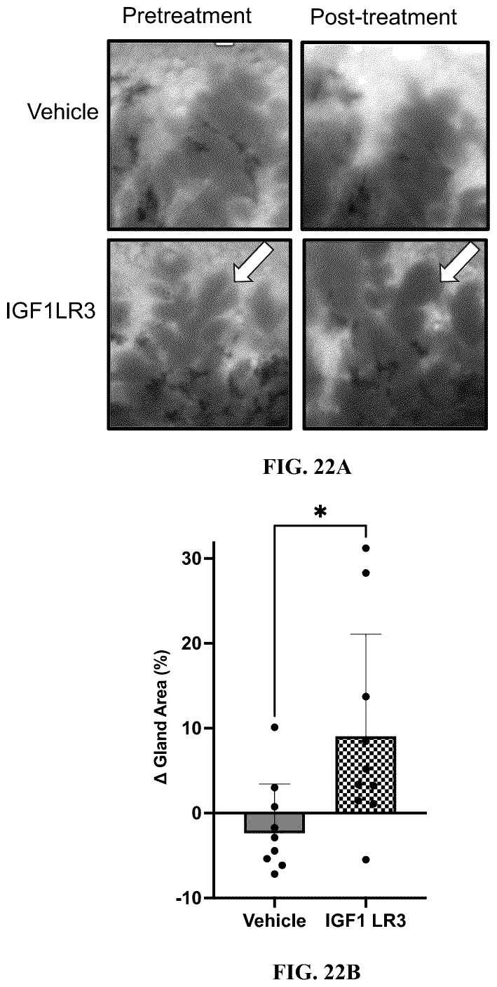

In FIGS. 22A and 22B, animals received a daily bilateral 10 μl dose of Vehicle (PBS) or 1 mg/ml IGF-1 LR3 (SEQ ID NO: 12) in each eye for 4 weeks.

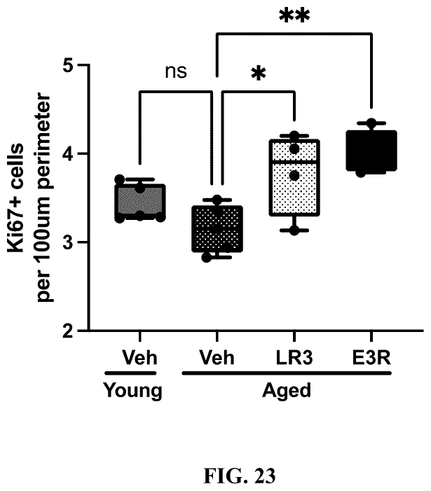

In FIG. 23, animals were administered a 10μl eyedrop containing equimolar concentration based on molecular weight of either IGF-1 LR3 (SEQ ID NO: 12) at 1.27 mg/ml or IGF-1 E3R (SEQ ID NO: 4) at 1 mg/ml every day for 2 weeks and sacrificed 24 hours after the last dose.

### Tissue Collection, Processing and Analysis

Mice were sacrificed by cervical neck dislocation after Isoflurane anesthesia and the upper left and right eyelids removed. Eyelids were then trimmed to the central 4 mm of tissue, embedded in Tissue-Tek® O.C.T. Compound (Sakura Finetek, Torrance, CA) and then snap frozen over liquid nitrogen. Tissue blocks were then sectioned, 8 μm thick, using a Leica CM 1850 cryostat (Leica, Wetzlar, Germany). Tissue sections were then stored in an ultra-low freezer until processed for fluorescent microscopy.

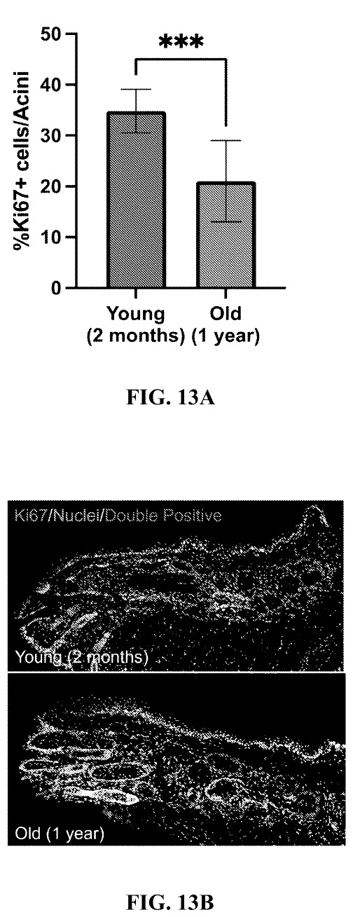

To measure basal proliferation rates or the effects of IGF-1LR3 on cell proliferation (FIGS. 13A, 13B, 14A, and 14B), tissue sections were stained with the cell proliferation marker, Ki67. Rabbit anti-Ki67 (Abcam, catalog #ab15580) were reacted with tissue sections for 1 hour at 37° C. in a humidified chamber. Sections were then washed in PBS (3×5 min) and secondary antibody (Goat Anti-Rabbit AlexaFluor546, Invitrogen) was applied to the sections for 1 hour and subsequently were rinsed with PBS (3×5 min), counterstained with DAPI (1:5000) and then mounted with a coverslip. Fluorescent imaging was performed on a Leica DMI6000B fully automated inverted fluorescence microscope (Leica Microsystems Inc., Buffalo Grove, IL) and tiled images over the eyelid tissue section and meibomian gland were collected using a low-light-level camera (QIClick, QImaging, British Columbia, Canada) and Leica 20× HC Plan Apo, 0.75 NA objective. Images were then stitched using Meta Imaging Series software. Quantification of Ki67 labeling was performed using QuPath, a bioimaging software analysis tool for high-throughput biomarker analysis. Initially, individual acini in each tissue section were annotated using the free-form region of interest tool, and the positive Ki67 stained nuclei within each acini identified using the positive cell detection tool. The number of Ki67 positive cells and the perimeter of each acinus was then recorded and the average number of label cells/100 μm of acinar perimeter calculated for each section. The average of three tissue sections was then determined the average for mouse calculated. Differences between treatment groups were then determined using a student's t-test (FIGS. 13A and 13B) or Two-Way Analysis of Variance, Tukey all pairwise multiple comparison procedures in both SigmaStat (Systat Software Inc., Point Richmond, CA) and GraphPad Prism (Insight Partners, NY, NY).

To assess changes in lipid synthesis (FIGS. 16A and 16B), cells were stained with the neutral lipid fluorescent probe, HCS LipidTox (Invitrogen, Carlsbad, CA). Cells were initially fixed in 2% paraformaldehyde in PBS and then rinsed and incubated in LipidTox solution (dilution 1:100) for 20 minutes at room temperature followed by DAPI staining. Fluorescent staining was then imaged using the Leica DMI6000B inverted microscope and tiled images over the eyelid section collected and stitched together using Meta Imaging software. To quantify newly synthesized lipid droplets near basal acinar cells, the region of lipid staining in the meibomian gland was segmented using the Threshold sub-routine to identify pixels with intensity >50. The threshold region was then extracted using the duplicate plane function and newly synthesized individual lipid droplets, which showed high intensity fluorescent staining, were identified using the count sub-routine set to identify particles greater than 2 μm and less than 5 μm in diameter that had a threshold intensity greater than 1000. Individual acini were then manually outlined using the free-hand region tool and the area of the acinus and number of lipid droplets/acinus recorded. The number of lipid droplets/acinus were then normalized to an average acinar area of 4000 μm². The average number of lipid droplets in three individual sections were then calculated, followed by the average number/mouse. The difference in droplets/acini numbers per treatment group were then determined using One-Way Analysis of Variance, all pairwise multiple comparison with Tukey's method GraphPad Prism (Insight Partners, NY, NY).

To evaluate activation of IGFIR1 by IGF-1 LR3 (FIG. 17), phospho-AKT levels were measured using the phospho-Akt (S473) Pan Specific DuoSet IC ELISA (RnD #DYC887B-2). Eyelid tissue was flash frozen on dry ice and stored at −80C until later analysis. Samples were weighed and homogenized 1 ml/50 mg tissue in T-PER (Thermo Scientific, 78510) containing protease and phosphatase inhibitors (Thermo Scientific, A32959 using a NextAdvance Bullet Blender Gold (BB24AU) with Green, Navy or Red Eppendorf Lysis Kits (NextAdvance). Homogenates were then used directly in the assay. ELISAs were performed in 384-well plates to the manufacturers' recommendation with minor modifications. Briefly, wells of 384-well plates were coated with 25 μl 6 μg/ml phospho-Akt1 (S473) capture antibody (#841692) in PBS (25 μl per 384-well), sealed and placed overnight at RT. Wells were washed 4× with 100 μl PBS containing 0.05% Tween 20, and blocked with 50 μl PBS containing 2% BSA for 1-2 h at room temperature (RT). Wells were washed 4× with 100 μl PBS containing 0.05% Tween 20 and incubated with 25 μl of lysate or P-AKT standard for 2 h at RT. Wells were washed 4× with 100 μl PBS containing 0.05% Tween 20 and then incubated with 25 μl 100 nanograms per milliliter (ng/ml) phospho-Akt1 (S473) detection antibody (#843081) diluted in PBS containing 1% BSA for 1 h at RT. Wells were washed 4× with 100 μl PBS containing 0.05% Tween 20 and incubated with 25 μl Streptavidin-HRP A (RnD #890803) diluted 1:200 in PBS containing 1% BSA for 20 min at RT. Wells were washed 4× with 100 μl PBS containing 0.05% Tween 20 and incubated with 25 μl TMB substrate solution (Abcam TMB ELISA Substrate High Sensitivity (ab171523; lot GR3427893-1)), and incubated for 10-20 min at RT. 12.5 μl stop solution (RnD #895926 from Ancillary Kit 2) was added. OD450 data for standard curves and each sample was collected using a Promega GloMAX Discover Microplate Reader. Sample data was interpolated from standard curves, corrected for dilution factors, plotted and pAKT levels were plotted and compared statistically using One-way ANOVA and Tukey's method for multiple comparisons in GraphPad Prism (Insight Partners, NY, NY).

### Mouse Eyelid Meibography

Transillumination meibography (FIGS. 15A and 15B) was conducted before the start of the study and just prior to sacrifice using techniques previously published. Briefly, mice were anesthetized using isoflurane and placed under a binocular dissection microscope (Leica MZ16FA, Leica Microsystems, Heerbrugg, Switzerland) equipped with a monochromatic camera (DFC340FX, Leica Microsystems, Heerbrugg, Switzerland). A broad band, halogen light source (OSL2 and OSL2B, Thorlabs, Newton, NJ) was used to transilluminate the eyelid for meibography. The light was delivered by a special optical fiber (BFL200HS02, Thorlabs, Newton, NJ) that had an input connector comprised of a bundle of seven 200 μm-diameter fibers and an output connector comprised of a linear array of fibers attached to a diffuser and a prism with glue. All images were taken at 20× magnification. After sacrifice, eyelids were removed and ex vivo meibography performed by placing eyelids onto an LED back light plate and taking transillumination photographs using the dissection microscope. To quantify meibomian gland area, individual eyelid images were analyzed using Meta Imaging Series software (Molecular Devices, Downington, PA). Specifically, the region of individual meibomian glands were manually outlined and the area of the region of interest measured for each individual gland in each image. The average area for the glands in each eyelid were then calculated and differences between the groups statistically analyzed using One-tailed Mann-Whitney test, using GraphPad Prism for (Insight Partners, NY, NY).

### Immunohistochemistry Staining (IHC)

Eyelids from Wild-type (WT) female C57BL/6J (Jackson Labs, Strain #000664) were dissected. Tissue was fixed in 4% paraformaldehyde overnight and switched to 30% sucrose 24 hours later. Eyelids were then embedded in OCT medium and sectioned in 10 micron sections and adhered to glass slides. Tissue was then stained for ki67 using Rat anti-Ki67 at 1 μg/mL (Invitrogen #14-5698-82) followed by detection with an anti-rat Alexafluor-labeled secondary antibody and IGFBP2 using Rabbit anti-IGFBP2 at 1 μg/mL (Abcam #ab 188200) followed by detection with an anti-rabbit Alexafluor-labeled secondary antibody. In between antibody incubations, slides were washed with PBS+0.1% Tween-20 3×. Slides were then coverslipped and imaged.

In FIGS. 21 and 23, animals were sacrificed as previously described, and eyelids were fixed in 4% paraformaldehyde overnight and switched to 30% sucrose 24 hours later. Eyelids were then embedded in OCT medium and sectioned in 10 micron sections and adhered to glass slides. Tissue was then stained for ki67 using Rat anti-Ki67 at 1 μg/mL (Invitrogen #14-5698-82) followed by detection with an anti-rat Alexafluor-labeled secondary antibody. In between antibody incubations, slides were washed with PBS+0.1% Tween-20 3×. Slides were then coverslipped and imaged. Cell nuclei were then stained with Hoescht and cover slipped for imaging. Random regions of interest (ROIs) were identified containing meibomian gland acini. Acini perimeter was outlined based on Hoescht stain in Image J and number of ki67+ cells lining the perimeter were measured and represented as number of ki67+ cells per 100 um of acini perimeter. Data was then quantified and analyzed in GraphPad Prism (Insight Partners, NY, NY).

### Results

As depicted in FIGS. 13A and 13B, young mice have increased proliferation in meibomian gland acini compared to old mice. Systemic IGF-1 LR3 treatment increased proliferation in meibomian gland acini in aged mice (FIGS. 14A and 14B); reversed atrophy and increased Meibomian glad area in aged mice (FIGS. 15A and 15B); increased lipid synthesis in Meibomian glands regardless of age (FIGS. 16A and 16B); and could be delivered systemically or by ocular drops to activate IGFIR in the eyelid (FIG. 17).

Next, the duration of effects of IGF-1 variations were investigated. IGF-1 LR3 (SEQ ID NO: 12) maintained significantly elevated pAKT (IGF1R activation) at 2 hours post dosing, whereas wild-type IGF-1 no longer showed significant pAKT at 2-hours post dosing, as shown in FIG. 18. IGF-1 LR3 (SEQ ID NO: 12) and another IGFBP-binding-deficient IGF-1 variant, IGF-1 Des1-3 (SEQ ID NO: 3), significantly elevated pAKT (IGF1R activation) at 2 hours post dosing compared to wild-type IGF-1, as shown in FIG. 19. It suggests that some IGF-1 variants with reduced affinity to IGFBPs prolong duration of IGF1R activation compared to wild-type IGF-1. Furthermore, IGF-1 LR3 (SEQ ID NO: 12) and IGF-1 E3R (SEQ ID NO: 4) have similar potency in vivo. IGF-1 LR3(SEQ ID NO: 12) or an IGFBP-binding-deficient IGF-1 variant IGF-1 E3R (SEQ ID NO: 4) increased pAKT (IGF1R activation) to levels comparable to IGF-1 at 1 hour post dosing, as shown in FIG. 20.

We next tested the effects of IGF-1 LR3 and IGF-1 ER3 in aged mouse meibomian glands. IGF-1 LR3 (SEQ ID NO: 12) induced dose-responsive proliferation in basal cells of the meibomian gland, as shown in FIG. 21. Two weeks of daily eyedrop dosing of IGF-1 LR3 (SEQ ID NO: 12) at the indicated concentration of 0.3, 1 or 3 mg/ml induced basal cell proliferation as determined by IHC, as shown in FIG. 21. IGF-1 LR3 (SEQ ID NO: 12) regenerated atrophied Meibomian glands in aged mice. Daily treatment with IGF-1 LR3 (SEQ ID NO: 12) for 1 month increased area of Meibomian glands comparing pre-and post-treatment, as shown in FIGS. 22A and 22B. IGF-1 LR3 (SEQ ID NO: 12) and IGF-1 E3R (SEQ ID NO: 4) induced proliferation in basal cells of the Meibomian gland. Two weeks of daily eyedrop dosing of IGF-1 LR3 (SEQ ID NO: 12) or IGF-1 E3R (SEQ ID NO: 4) induced basal cell proliferation in aged mice as determined by IHC, as shown in FIG. 23.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating dry eye disorder or Meibomian gland dysfunction in a subject in need thereof, the method comprising locally administering to an eye or eyelid of the subject a pharmaceutical composition comprising a therapeutically effective amount of a polypeptide consisting of an IGF-1 variant having at least 90% sequence identity to IGF-1 (SEQ ID NO:1), wherein the local administration of the pharmaceutical composition to the eye or eyelid results in an increase of lipid content of the Meibomian glands of the subject.

2. The method of claim 1, wherein the IGF-1 variant has reduced affinity to at least one IGF binding protein relative to wild-type IGF-1 to the IGFBP.

3. The method of claim 1, wherein the IGF-1 variant comprises an amino acid substitution at position 3 relative to wild-type IGF-1 (SEQ ID NO: 1), and the position numbering is based on alignment of the IGF-1 variant to SEQ ID NO: 1 with the positions being numbered from an N-terminus of SEQ ID NO: 1 to a C-terminus of SEQ ID NO: 1.

4. The method of claim 1, wherein the local administration is performed daily.

5. The method of claim 1, wherein the local administration is performed via an eyedropper.

6. The method of claim 1, wherein the pharmaceutical composition is administered to an outer eyelid of the subject.

7. The method of claim 1, wherein the pharmaceutical composition is a cream.

8. The method of claim 1, wherein the locally administering the pharmaceutical composition to the subject results in an increase in surface area or volume of meibomian glands within the inner eyelid surface of the subject.

9. The method of claim 1, wherein the locally administering the pharmaceutical composition to the subject results in an increase in lipid content within a Meibomian gland of the subject.

10. The method of claim 1, wherein the locally administering the pharmaceutical composition to the subject results in an increase in release of lipid from acini of a Meibomian gland of the subject.

11. The method of claim 1, wherein the locally administering the pharmaceutical composition to the subject results in an increase in duration of phosphorylation of Akt in meibocytes.

12. The method of claim 1, wherein the locally administering the pharmaceutical to the subject results in an increase in a size of the meibomian glands.

13. The method of claim 1, wherein the locally administering the pharmaceutical to the subject results in a decrease in meibomian gland atrophy.

14. The method of claim 1, wherein the locally administering the pharmaceutical to the subject results in a reversal of age-associated meibomian gland atrophy.

15. The method of claim 1, wherein the locally administering the pharmaceutical to the subject results in an increase in corneal epithelial cell proliferation.

16. The method of claim 1, wherein the locally administering the pharmaceutical to the subject results in an increase in corneal healing

17. The method of claim 1, wherein the locally administering the pharmaceutical to the subject results in an increase in IGF1 receptor (IGF1R) activation in the meibomian glands.

18. The method of claim 1, wherein the method does not comprise administration of any additional phospholipidosis-inducing agent.

19. The method of claim 1, wherein the method does not comprise administration of one or both of azithromycin and doxycycline.

20. The method of claim 1, wherein the pharmaceutical composition comprises one or more pharmaceutically acceptable excipients selected from water, saline, sucrose, lactose, malic acid, cellulose sugar, mannitol, maltitol, dextran, sorbitol, starch, agar, alginate, chitin, chitosan, pectin, tragacanth gum, gum arabic, gelatin, collagen, casein, albumin, synthetic or semi-synthetic polymer or glyceride, methyl cellulose, hydroxypropylmethyl-cellulose, polyvinylpyrrolidone, or any combination thereof.

21. The method of claim 1, wherein the IGF-1 variant has at least 95% sequence identity to SEQ ID NO: 1.

22. The method of claim 1, wherein the IGF-1 variant has at least 97% sequence identity to SEQ ID NO: 1.

23. A pharmaceutical composition formulated for local administration, the pharmaceutical composition comprising a therapeutically effective amount of a polypeptide consisting of an IGF-1 variant having a sequence with at least 95% sequence identity to wild-type IGF-1 (SEQ ID NO: 1), wherein the polypeptide has reduced affinity to at least one IGF binding protein (IGFBP) as compared to the affinity for the interaction between wild-type IGF-1 (SEQ ID NO: 1) and the IGFBP.


SEQUENCE LISTING (13 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 70 aa, synthetic construct
GPETLCGAELVDALQFVCGDRGFYFNKPTGYGSSSRRAPQTGIVDECCFRSCDLRRLEMLCAPLKPAKSA

SEQ ID NO: 3 - protein, 67 aa, synthetic construct
TLCGAELVDALQFVCGDRGFYFNKPTGYGSSSRRAPQTGIVDECCFRSCDLRRLEMYCAPLKPAKSA

SEQ ID NO: 4 - protein, 70 aa, synthetic construct
GPRTLCGAELVDALQFVCGDRGFYFNKPTGYGSSSRRAPQTGIVDECCFRSCDLRRLEMYCAPLKPAKSA

SEQ ID NO: 5 - protein, 69 aa, synthetic construct
GPETLCGAELVDALQFVCGDRGFYFNKPTGYGSSSRAPQTGIVDECCFRSCDLRRLEMYCAPLKPAKSA

SEQ ID NO: 6 - protein, 66 aa, synthetic construct
TLCGAELVDALQFVCGDRGFYFNKPTGYGSSSRAPQTGIVDECCFRSCDLRRLEMYCAPLKPAKSA

SEQ ID NO: 7 - protein, 105 aa, synthetic construct
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 8 - protein, 77 aa, synthetic construct
MRAAAPAVAATLCGAELVDALQFVCGDRGFYFNKPTGYGSSSRRAPQTGIVDECCFRSCDLRRLEMYCAPLKPAKSA

SEQ ID NO: 9 - protein, 10 aa, synthetic construct
MRAAAPAVAA

SEQ ID NO: 10 - protein, 15 aa, synthetic construct
CGRKKRRQRRRPPQC

SEQ ID NO: 11 - protein, 9 aa, synthetic construct
RRRRRRRRR

SEQ ID NO: 12 - protein, 83 aa, synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 13 - protein, 13 aa, synthetic construct
MFPAMPLLSLFVN